SEQUENCE LISTING (19 sequences)

SEQ ID NO: 1 - protein, 1057 aa, Arabidopsis thaliana
MNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLLGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKSIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKETKEKTNLEWHLPILAMTADVIHATYEECLKSGMDGYVSKPFEEENLYKSVAKSFKPNPISPSS

SEQ ID NO: 2 - protein, 131 aa, Arabidopsis thaliana
RPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDV

SEQ ID NO: 3 - protein, 144 aa, Arabidopsis thaliana
RPLTSGVAYALKVPHSEREKFEKEHGWAIKKMETEDQTVVQDCVPENFDPAPIQDEYAPVIFAQETVSHIVSVDMMSGEEDRENILRARASGKGVLTSPFKLLKSNHLGVVLTFAVYDTSLPPDATEEQRVEATIGYLGASYDM

SEQ ID NO: 4 - protein, 146 aa, Arabidopsis thaliana
RPLTSGVAYAMRVLHSEREEFERQQGWTIRKMYSLEQNPVHKDDYDLEALEPSPVQEEYAPVIFAQDTVSHVVSLDMLSGKEDRENVLRARSSGKGVLTAPFPLIKTNRLGVILTFAVYKRDLPSNATPKERIEATNGYLGGVFDI

SEQ ID NO: 5 - protein, 156 aa, Populus trichocarpa
RPLTSGVAYAVRVMHSEREQFENQQGWTIKRMDTFEQSPVQKDDNVAKALEPSPIQEEYAPVIFAQDTVAHVVSLDMLSGTEDRENVLRARASGKGVLTAPFRLLKTNRLGVILTFAVYKTDLPSNAMPNERIQATDGSSDLLMTPIRYLGGIFDI

SEQ ID NO: 6 - protein, 146 aa, Vitis vinifera
RPLTSGVAYAVRVLHSEREQFEKQQGWTIKRMDTPEQTPVHEDNHASENLEPSPVQEEYAPVIFAQDTVSHVISLDMLSGKEDRENVLRARASGKAVLTAPFRLFKTNSLGVILTFAVYKSDLLSNATPNERIQATHGYLGGVFHI

SEQ ID NO: 7 - protein, 130 aa, Oryza sativa
RPLTSGLAYAARVTHSERELFERQQAWSIRAMNFSSKRPRAEEYAPVIFAQDAYKHVVSIDMLSGAEDRGNLLRARESGKVVLTAPFQLLNKRIGVVLTYAVYKSELPLNATVHDRIQSSIGYLGGVFDI

SEQ ID NO: 8 - protein, 130 aa, Lotus japonicus
RPLMSGVAYAQRVVHSERERFEKQHGWVIKTMERVPSGVRDEYAAVIFAQETVSYLESIDMMSGEEDRENILRARATGKAVLTSPFRLLDSHHLGVVLTFPVYKSKLPPEPTTEEVIKAIAGYIGGSFDV

SEQ ID NO: 9 - protein, 129 aa, Zea mays
RPLLSGVAYAQRVVHADREGFERHQGWIIKTMKHEPSPAQDEYAPVVYSQETVSYIEGLDMMSGEEDRENILRSRASGKAVLTRPFRLMSNHLGVVLTFPVYHADLPSDAKEEDRVAATAGYLGGAFDV

SEQ ID NO: 10 - protein, 131 aa, Physcomitrella patens
RPLMSGVAYAERVLHANRNLFESKHGWTIKEMFSKEKQRDFDEYAPTTMTQETLSHLTSLDMMSGQEDRENIIRARESGKGALTNPFRLLESKHLGVVFTFAVYTTDLPEDATKEDRIKATAGYLGGAFDV

SEQ ID NO: 11 - protein, 70 aa, Arabidopsis thaliana
MDDANKIRREEVLVSMCDQRARMLQDQFSVSVNHVHALAILVSTFHYHKNPSAIDQETFAEYTARTAFER

SEQ ID NO: 12 - protein, 70 aa, Arabidopsis thaliana
TNEEIIMKRRETLANMCDERARVLQDQFNVSLNHVHALSILVSTFHHGKIPSAIDQRTFEEYTERTNFER

SEQ ID NO: 13 - protein, 70 aa, Arabidopsis thaliana
FSSQAMEKRKETLASMCDERARMLQDQFNVSMNHVQAMSILISTFHHGKIPSAIDQRTFSEYTDRTSFER

SEQ ID NO: 14 - protein, 70 aa, Populus trichocarpa
MSSQAFEKRKETLASMCDERARMLQDQFNVSMNHVQAMSILISTFHHGKNPSAIDQRTFARYTERTAFER

SEQ ID NO: 15 - protein, 70 aa, Vitis vinifera
LSLQASEKRKETLGSMCDERARMLQDQFNVSMNHVQAMSILISTFHHGKNPSAIDQGTFARYTERTAFER

SEQ ID NO: 16 - protein, 70 aa, Oryza sativa
MRSQATEARHGTLAVMCDERARILQDQVKVSMNHLQALAILVSTFHHSKSPSAIDQTTFARYVERTAFER

SEQ ID NO: 17 - protein, 70 aa, Lotus japonicus
MDAETKVRRKEVLGSLCDQRARMLQDQFSVSVNHVHALAILVSTFHYYRNTSAIDQETFAEYTARTAFER

SEQ ID NO: 18 - protein, 70 aa, Zea mays
FRHAALRKAEEALVSMCEERARMLQDQFAVSVNHVHALAILVATFHYEKHPPALDQNTFADYTARTSFER

SEQ ID NO: 19 - protein, 70 aa, Physcomitrella patens
MRFDMLERRRENLANMCEGRARMLEEQFKASMNHVRALTALLTTFHLGKQPSAINQETFATYAERTAFER

(19) United States
(12) Patent Application Publication
(10) Pub. No.: US 2013/0004966 A1
(43) Pub. Date: Jan. 3, 2013

(54) STRUCTURE OF CYTOKININS AND CYTOKININ RECEPTORS, AND MODULATION OF CYTOKININ SIGNALING

(75) Inventors: Michael Hothorn, Lauenau (DE); Tsegaye Dabi, San Diego, CA (US); Joanne Chory, Del Mar, CA (US)

(73) Assignee: Salk Institute for Biological Studies (027731), La Jolla, CA (US)

(21) Appl. No.: 13/475,021

(22) Filed: May 18, 2012

Related U.S. Application Data
(60) Provisional application No. 61/488,341, filed on May 20, 2011.

(51) Int. Cl. C12N 9/12; G01N 33/53

(52) U.S. Cl. 435/7.8; 435/194

(57) ABSTRACT

Provided herein are seven high resolution crystal structures showing the cytokinin receptor AHK4 complexed with various naturally occurring and synthetic ligands. Further provided are strategies for modulating cytokinin receptor activity.

### CROSS-REFERENCES TO RELATED APPLICATIONS

The present application claims priority to U.S. Provisional Patent Application No. 61/488,341 filed May 20, 2011, the disclosure of which is incorporated herein by reference in its entirety.

### STATEMENT AS TO RIGHTS TO INVENTIONS MADE UNDER FEDERALLY SPONSORED RESEARCH AND DEVELOPMENT

The invention was made with Government support under Grant Nos. 5R01GM52413 and P30 NS057096 awarded by the National Institutes of Health, and Grant No. 10S-0649389, awarded by the National Science Foundation. The Government has certain rights in the invention.

### BACKGROUND OF THE INVENTION

Plants have evolved hormone signaling pathways to integrate growth, development and interactions with the environment. These signaling cascades are initiated in the cytoplasm, nucleus and at membranes, where two-component sensor histidine kinases (HKs) perceive the phytohormones ethylene (Chang et al., Science 262:539-544 (1993)) and cytokinin (Mähönen et al., Genes Dev. 14:2938-2943 (2000); Inoue et al., Nature 409:1060-1063 (2001); Suzuki et al., Plant Cell Physiol. 42:107-113 (2001); Ueguchi et al., Plant Cell Physiol. 42:751-755 (2001)).

Cytokinins are classic, adenine-based phytohormones that orchestrate plant growth, development and, in concert with other plant hormones, the integrity of stem cell populations (Perilli et al., Current Opinion in Plant Biology 13:21-26 (2010); Stahl & Simon, Curr. Opin. Plant Biol. 13:53-58 (2010); Zhao. et al., Nature 465:1089-1092 (2010)). Plant cytokinin receptors respond to a variety of adenine-based natural cytokinins as well as to urea-based synthetic cytokinins (Chory & Kieber, Trends Plant Sci. 13:85-92 (2008)), yet how these receptors evolved and how they perceive chemically diverse naturally occurring and synthetic cytokinins with high specificity is unknown. Cytokinin receptors contain a ˜270 residue sensor domain that is sandwiched between two trans-membrane helices TM1 and TM2. Mutation of the conserved Thr278 in the sensor domain to Ile leads to a loss-of-function phenotype. TM2 is connected to a cytoplasmic HK that consists of an N-terminal dimerization domain and a C-terminal catalytic domain (Gao & Stock, Annu. Rev. Microbiol. 63:133-154 (2009)). Cytokinin binding to the sensor domain results in phosphorylation of a conserved His residue in the dimerization domain (Heyl & Schmülling, Curr. Opin. Plant Biol. 6:480-488 (2003)). This phosphoryl group is transferred via an Asp residue in a C-terminal receiver domain to a downstream histidine phosphotransfer protein, and further to a response regulator. While the cellular and developmental roles of the three Arabidopsis cytokinin receptors have been studied (Riefler et al., Plant Cell 18:40-54 (2006); Higuchi et al., Proc. Natl. Acad. Sci. U.S.A. 101:8821-8826 (2004)), little is known about how these proteins can recognize chemically diverse cytokinins.

Provided herein is the crystal structures of the Arabidopsis histidine kinase 4 (AHK4) sensor domain, which exhibits significant structural homology with a family of bacterial histidine kinases. Cytokinin binding to a small PAS-like domain of the receptor results in structural changes that may ultimately cause movements of the trans-membrane helices, thus signaling the loading state of the sensor to the cytoplasmic kinase domain. Several complex structures and mutational analyses reveal a highly adaptable ligand binding pocket that accommodates diverse adenine- and urea-derived cytokinins. The disclosed structures define chemical restraints that may spur the design of novel synthetic cytokinins with potential applications in agriculture, and establish a strong evolutionary link between pro- and eukaryotic histidine kinases.

### BRIEF SUMMARY OF THE INVENTION

Provided herein are seven high resolution crystal structures of the isolated sensor domain of Arabidopsis histidine kinase 4 (AHK4), one of the 3 cytokinin receptors found in Arabidopsis. The results show that eukaryotic HKs are more closely related to bacterial sensor HKs, in particular bacterial family 1 sensor HKs, than was predicted based on sequence comparisons.

Accordingly, in some embodiments, provided herein is an isolated protein comprising a 3-dimensional crystal structure of a cytokinin receptor sensor domain as structurally defined by the atomic coordinate data shown in Tables 1 and 2. In some embodiments, the isolated protein comprises a 3-dimensional structure of the cytokinin receptor sensor domain, with a space group P32 2 1 and unit cell dimensions a=60.0±0.2 angstrom, b=60.0±0.2 angstrom, c=297.8±0.5, with alpha=90, beta=90, and gamma=120. In some embodiments, the isolated protein comprises a 3-dimensional structure of the cytokinin receptor sensor domain as structurally defined by the diagrams shown in FIGS. 1, 2, and 9. In some embodiments, the protein comprises Arabidopsis Histidine Kinase 4 (AHK4) or a species homolog of the AHK4 sensor domain. In some embodiments, the protein comprises a sequence having at least 90% identity to residues 126-391 of SEQ ID NO: 1. In some embodiments, the protein binds a cytokinin selected from the group consisting of N⁶-isopentyl adenine (iP), N⁶-benzyladenine (BA), trans-zeatin (tZ), kinetin (KIN), and thidiazuron (TD). In some embodiments, the protein homodimerizes.

In some embodiments, provided are methods for identifying (screening for) a candidate modulator of cytokinin receptor (e.g., AHK4), comprising
(a) comparing the structure of a test compound with the structure of cytokinin receptor, said cytokinin receptor comprising a 3 dimensional structure selected from the group consisting of:
   (i) a sensor domain structurally defined by the atomic coordinate data shown in Tables 1 and 2;
   (ii) a space group P32 2 1 and unit cell dimensions a=60.0±0.2 angstrom, b=60.0±0.2 angstrom, c=297.8±0.5, with alpha=90, beta=90, and gamma=120; and
   (iii) a sensor domain structurally defined by the diagrams shown in FIGS. 1, 2, and 9;
(b) determining whether the test compound is likely to interact with cytokinin receptor; and
(c) identifying a candidate cytokinin receptor modulator when the test compound in step (b) is determined to be likely to interact with cytokinin receptor.

In some embodiments, the method further comprises validating the candidate cytokinin receptor modulator by contacting the candidate cytokinin receptor modulator with a cytokinin receptor (e.g., AHK4) and detecting interaction of the candidate cytokinin receptor modulator with cytokinin receptor. In some embodiments, the method further comprises detecting an effect of the candidate cytokinin receptor modulator when contacted with a cytokinin receptor expressing plant, wherein the effect is selected from the group consisting of: speeding leaf senescence, delaying leaf senescence, speeding seed germination, delaying seed germination,increasing shoot and root length, and reducing shoot and root length, as compared to a standard control. In some embodiments, the candidate cytokinin receptor modulator interacts with the ligand binding region of AHK4 (see FIGS. 1, 2, and 9). In some embodiments, the candidate cytokinin receptor modulator interacts with the dimerization region of AHK4 (FIGS. 4 and 5).

In some embodiments, provided are methods for identifying (screening for) a candidate modulator of a cytokinin receptor (e.g., AHK4), comprising
(a) comparing the structure of a test compound with the structure of cytokinin receptor, said cytokinin receptor comprising a 3 dimensional structure selected from the group consisting of:
   (i) a sensor domain structurally defined by the atomic coordinate data shown in Tables 1 and 2;
   (ii) a space group P32 2 1 and unit cell dimensions a=60.0±0.2 angstrom, b=60.0±0.2 angstrom, c=297.8±0.5, with alpha=90, beta=90, and gamma=120; and
   (iii) a sensor domain structurally defined by the diagrams shown in FIGS. 1, 2, and 9;
(b) detecting interaction of the test compound with cytokinin receptor, thereby identifying a candidate modulator of cytokinin receptor.

In some embodiments, the method further comprises rational design of the test compound, e.g., based on the AHK4 structure described herein. For example, prior to step (a), the structure of a test compound can be compared to the structure of AHK4 to determine the likelihood of interaction between the test compound and AHK4. In some embodiments, the method further comprises detecting an effect of the candidate cytokinin receptor modulator when contacted with a AHK4 expressing plant, wherein the effect is selected from the group consisting of: increasing or decreasing plant biomass and increasing or decreasing the size of vegetative structures in the plant, as compared to a standard control. In some embodiments, the candidate cytokinin receptor modulator interacts with the ligand binding region of cytokinin receptor (see FIGS. 1, 2, and 9). In some embodiments, the candidate cytokinin receptor modulator interacts with the dimerization region of cytokinin receptor (e.g., FIGS. 4 and 5).

Also provided are cytokinin receptor modulators (e.g., modulators of AHK2, AHK3, AHK4, and species homologs thereof). In some embodiments, the cytokinin receptor modulator is identified according to a method as described above. In some embodiments, the cytokinin receptor modulator is cytokinin mimetic identified as likely to bind the cytokinin binding site of the cytokinin receptor as characterized herein (e.g., in FIG. 9). In some embodiments, the cytokinin receptor modulator interacts with cytokinin receptor in the same way as a cytokinin (i.e., at the same residues and/or with the same affinity). In some embodiments, the cytokinin receptor modulator interacts with cytokinin receptor in a manner that is distinct from a cytokinin (i.e., at different residues and/or with a higher or lower affinity). In some embodiments, the cytokinin receptor modulator is modified, e.g., with a label, or to improve stability, using the cytokinin receptor structure described herein to ensure that the modification does not interfere with cytokinin receptor interactions.

In some embodiments, the cytokinin receptor modulator is a cytokinin receptor inhibitor. In some embodiments, the cytokinin receptor inhibitor interferes with, e.g., ligand binding to cytokinin receptor or dimerization of cytokinin receptor. Such inhibitors can be designed using the cytokinin receptor structural data disclosed herein for AHK4, e.g., to target cytokinin receptor residues critical for ligand binding or dimerization (see, e.g., FIGS. 1, 2, 4, 5, and 9). In some embodiments, the cytokinin receptor inhibitor, when contacted with a plant expressing cytokinin receptor, delays leaf senescence, speeds seed germination and/or increases the shoot and root length as compared to a standard control (e.g., a cytokinin receptor expressing plant in the absence of the inhibitor).

In some embodiments, the cytokinin receptor modulator is a cytokinin receptor agonist. Such agonists can be designed using the cytokinin receptor structural data disclosed herein for AHK4, e.g., to target cytokinin receptor residues involved in binding to the natural or synthetic cytokinins and dimerization (see, e.g., FIGS. 1, 2, 4, 5, and 9). In some embodiments, the cytokinin receptor agonist mimics or improves the binding of a cytokinin to cytokinin receptor. In some embodiments, the cytokinin receptor agonist stabilizes the dimerization domain. In some embodiments, the cytokinin receptor agonist, when contacted with a plant expressing cytokinin receptor, speeds leaf senescence, delays seed germination and/or reduces the size of shoots and roots, as compared to a standard control (e.g., a cytokinin receptor expressing plant absent the agonist).

### BRIEF DESCRIPTION OF THE DRAWINGS

FIG. 1. AHK4 binds cytokinins with its membrane-distal PAS domain. a,

Ribbon diagram of the sensor domain (residues 126-391). The N-terminal stalk helix and the dimerization interface is shown in orange, the membrane-distal cytokinin binding domain in dark-blue, and the membrane-proximal PAS domain is in light-blue, respectively. Disulfide bridges are depicted in green. One molecule of iP (in yellow) binds per AHK4 monomer. b, Close-up of the cytokinin binding pocket complexed with iP (in bonds representation). The ‘lid’ is shown in magenta. c, Cytokinin induced β-galactosidase activity in E. coli cells harboring wild-type and mutant forms of full-length AHK4. Only the WT, Y250E, M256A, and T294A show any β-galactosidase activity, indicating that the other indicated residues are required for cytokinin signaling.

FIG. 2. The cytokinin binding pocket in AHK4. a, Stereo close-up view of the membrane-distal PAS domain in AHK4 (in blue) in complex with iP (in yellow, in bonds representation). The contacting residues are labeled, the ‘lid’ at the entry of the binding pocket is highlighted in magenta. b, Structure based sequence alignment of the sensor domain in different cytokinin receptors: AHK4, Arabidopsis histidine kinase 4, Arabidopsis thaliana (UniProt Acc. Q9SIT0), AHK2, Arabidopsis thaliana (Q9FKH3), AHK3, Arabidopsis thaliana (Q9C5U1), PtHK, Populus trichocarpa (B9GZP2), VvHK, Vitis vinifera (D7TAZ7), OsHK, Oryza saliva (Q6PX60), LjHK, Lotus japonicus (A0PEY0), ZmHK, Zea mays (Q2ACB8), PpHK, Physcomitrella patens (Q27PX4). Residues interacting with iP are highlighted, invariant residues are labeled with a *, residues that were mutated in this study are highlighted by a red dot. The alignment includes a secondary structure assignment for AHK4 calculated with DSSP.

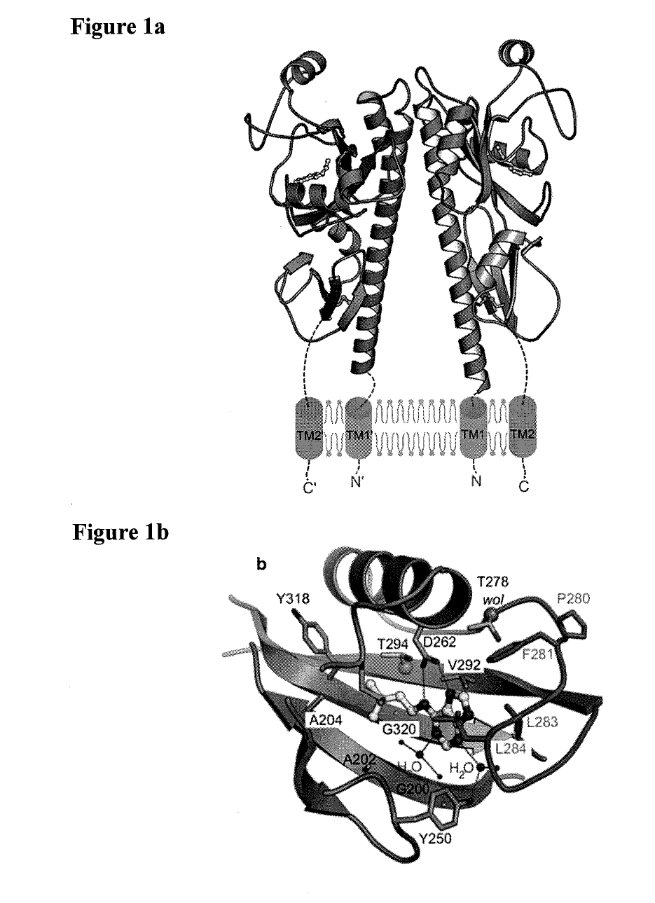

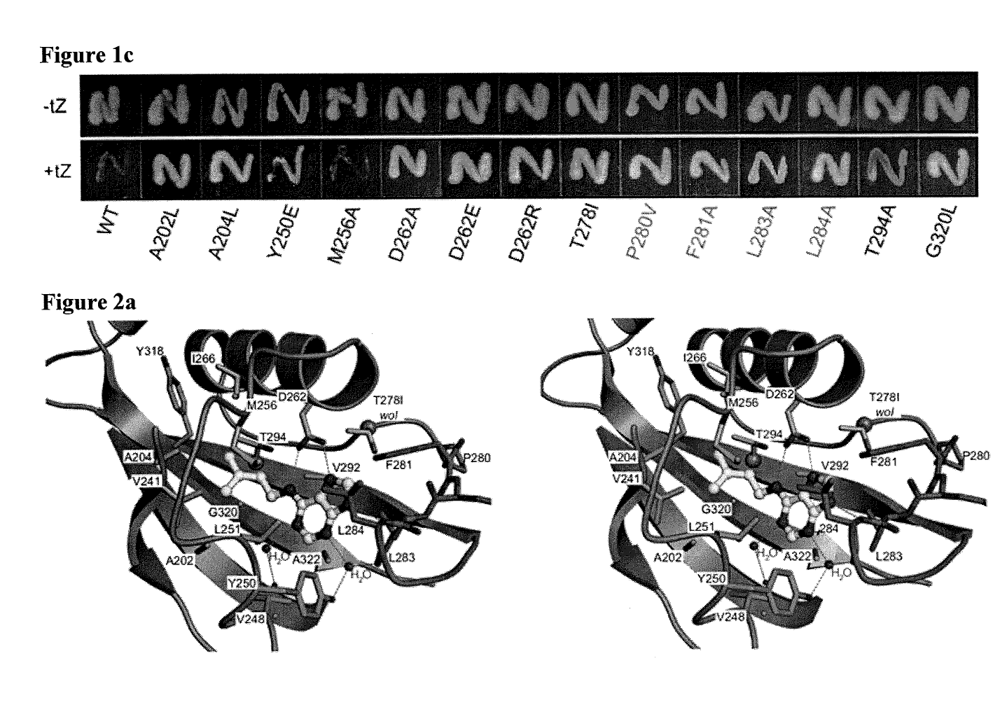

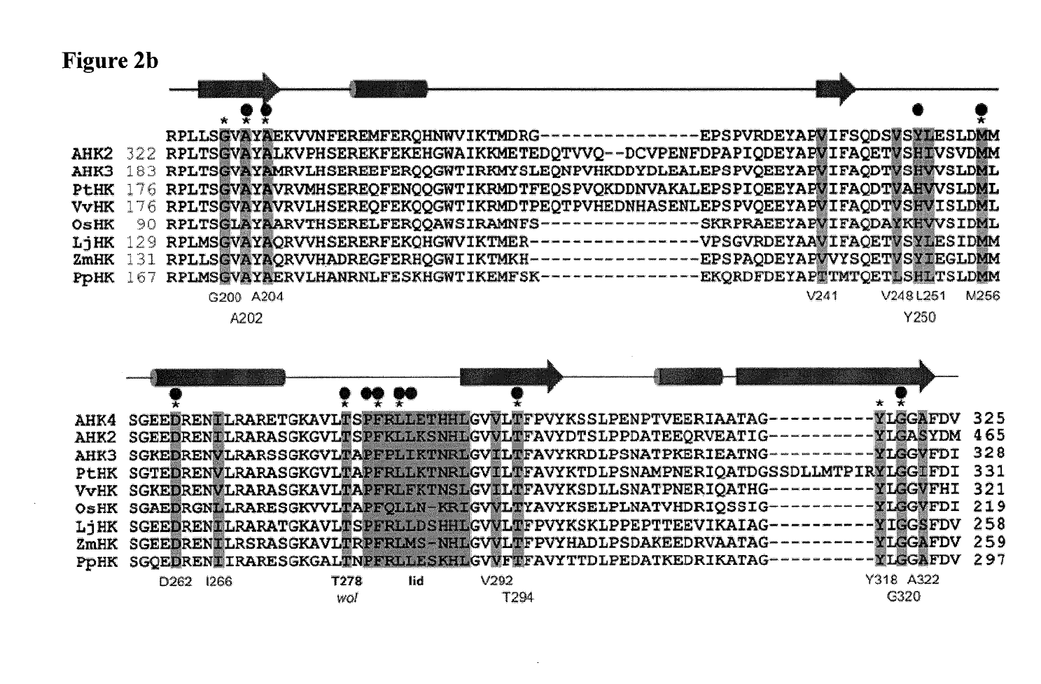

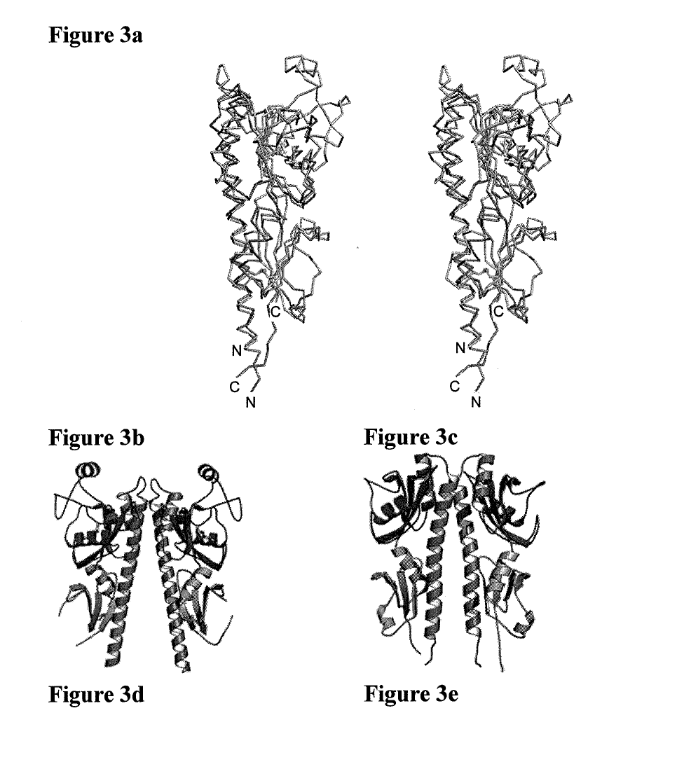

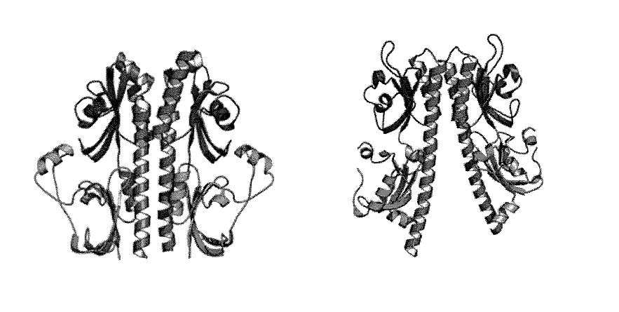

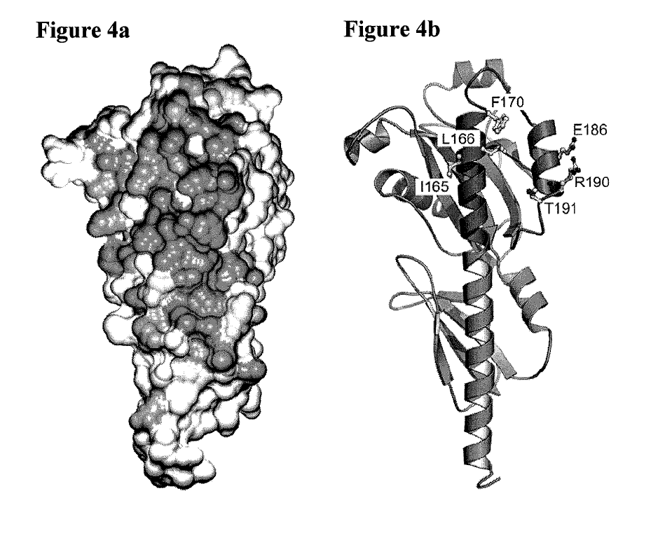

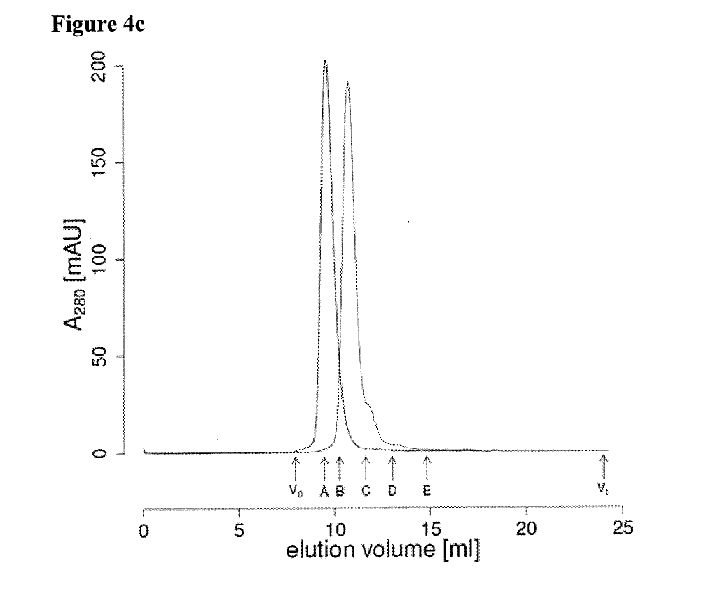

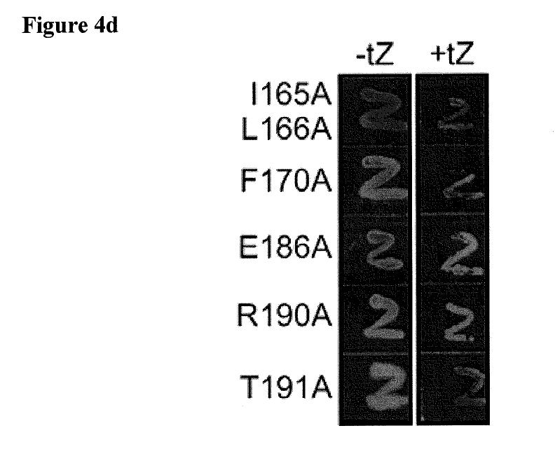

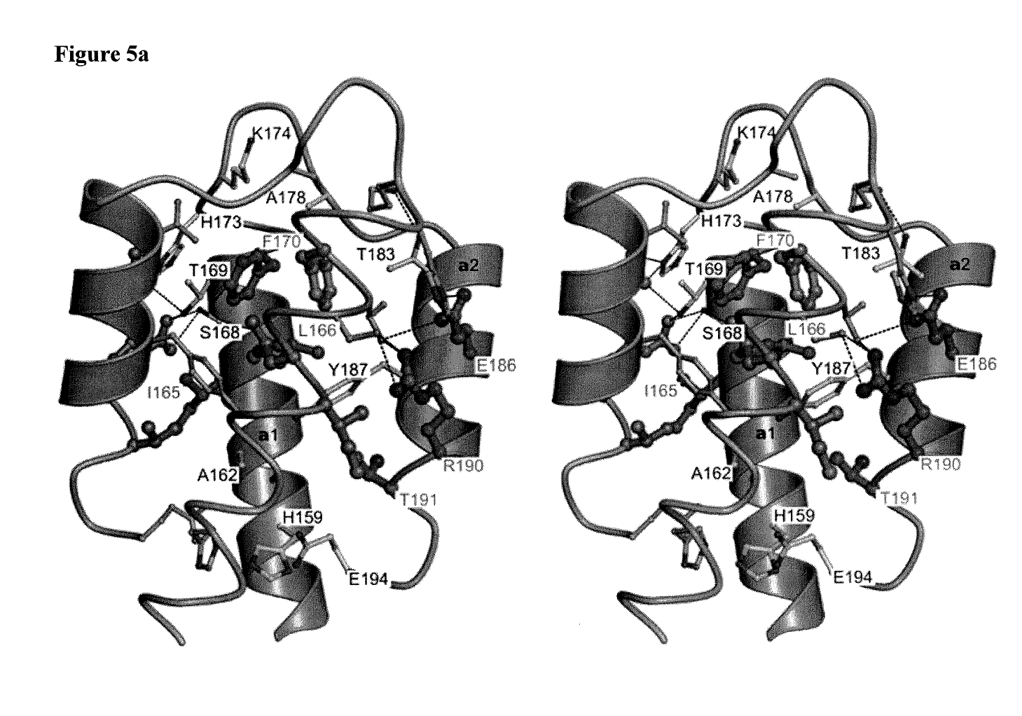

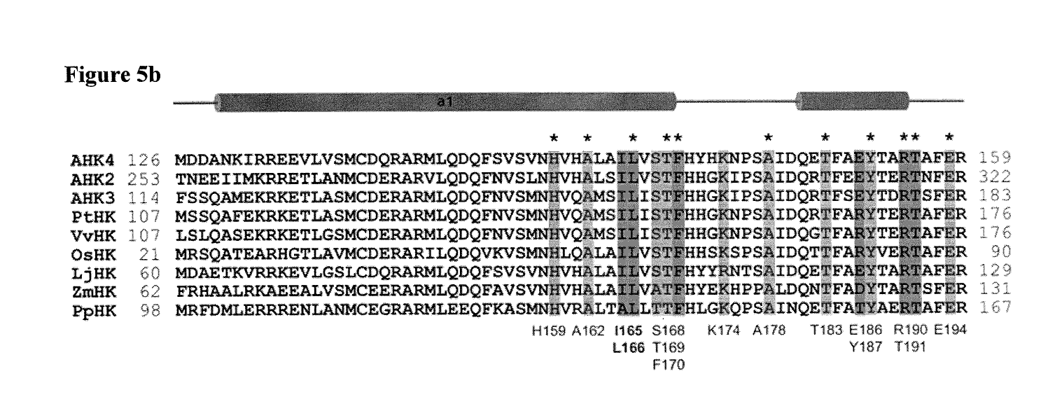

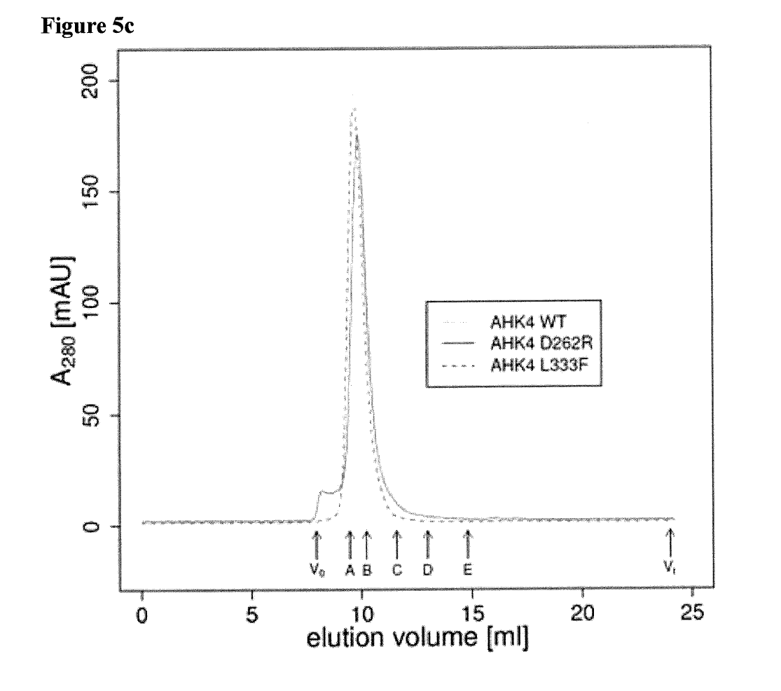

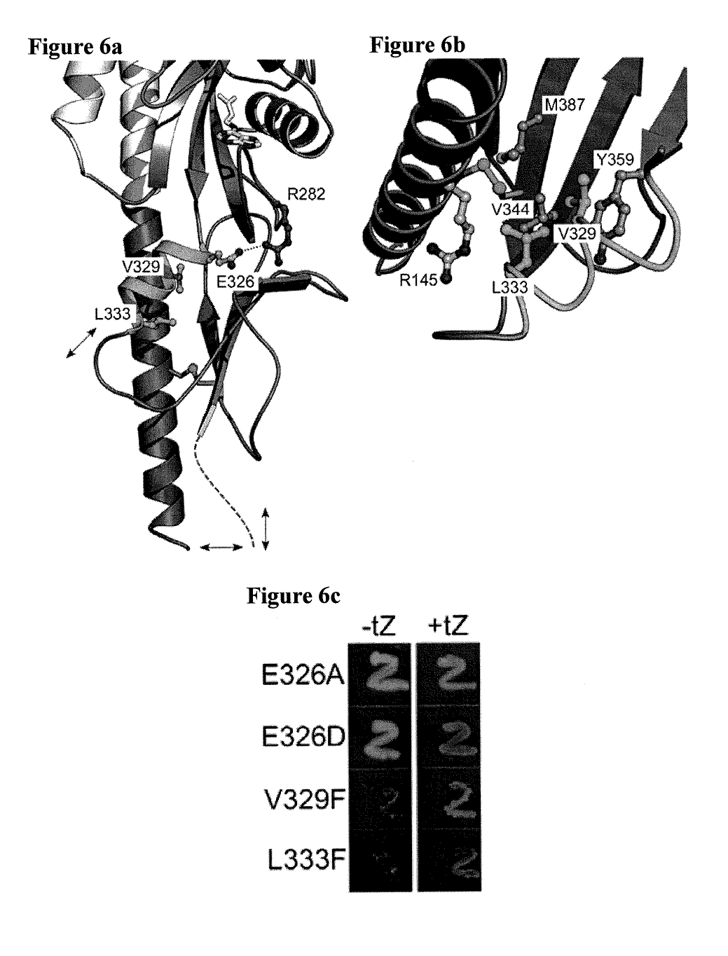

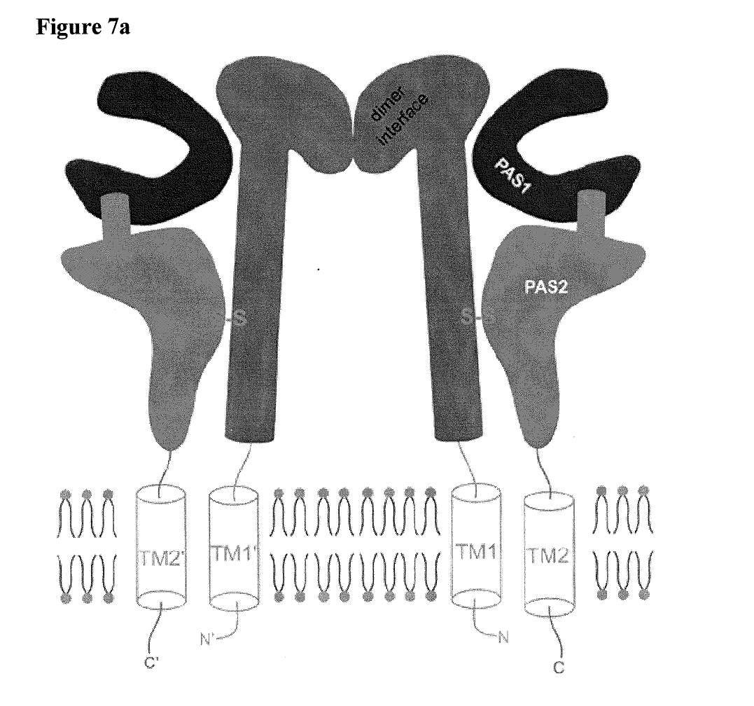

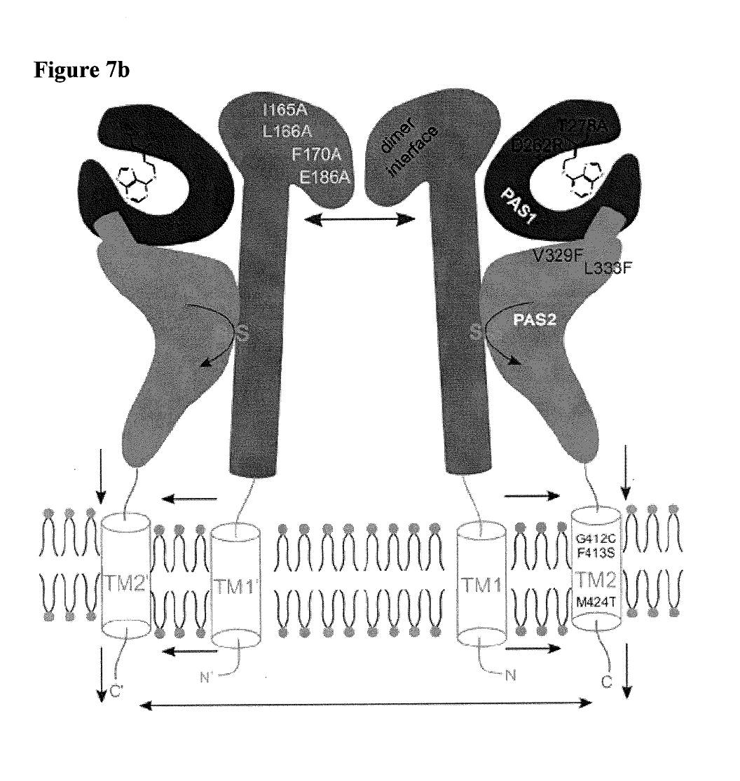

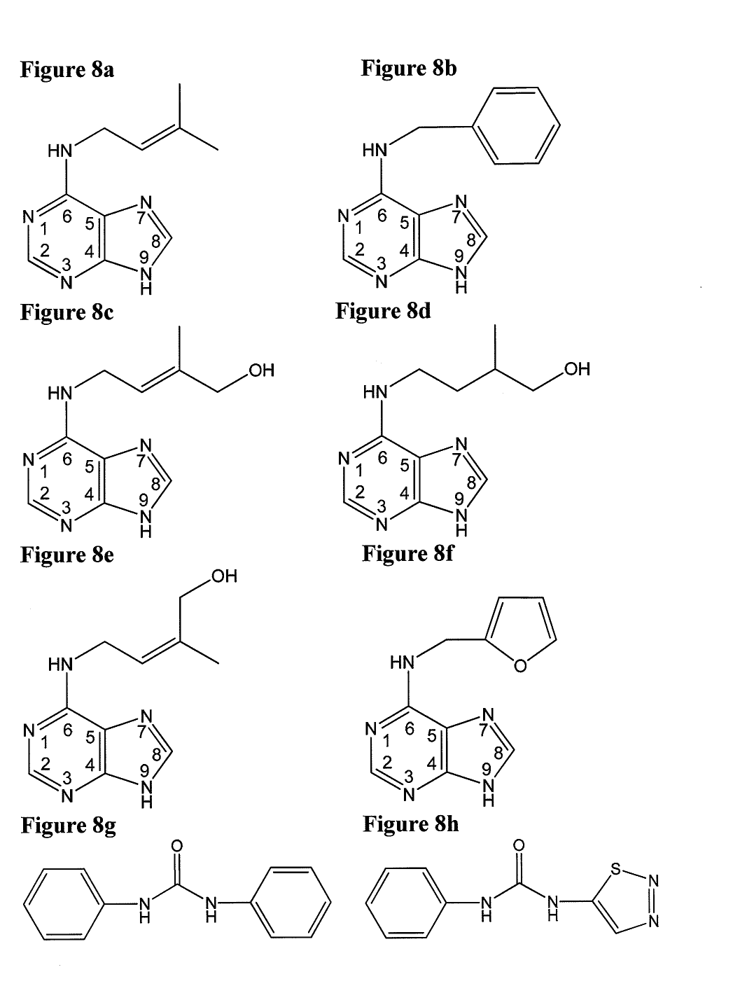

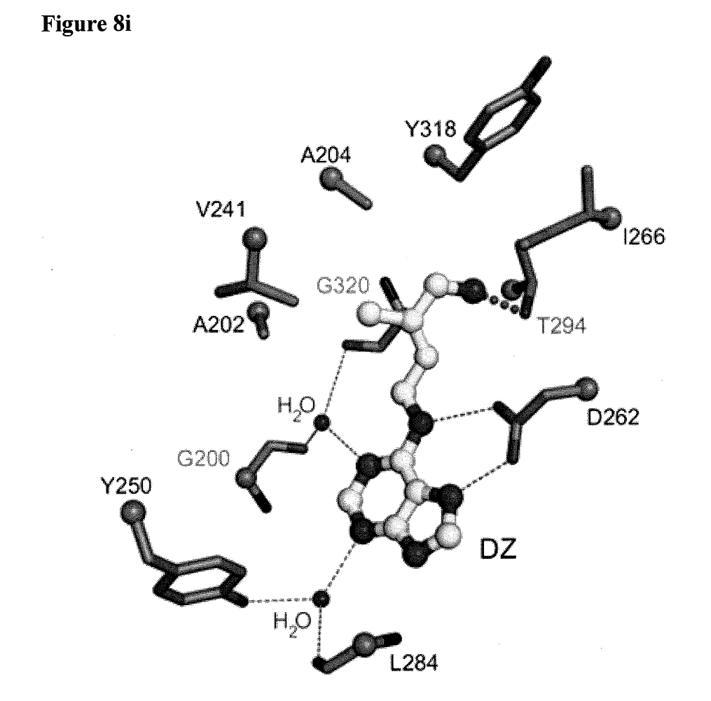

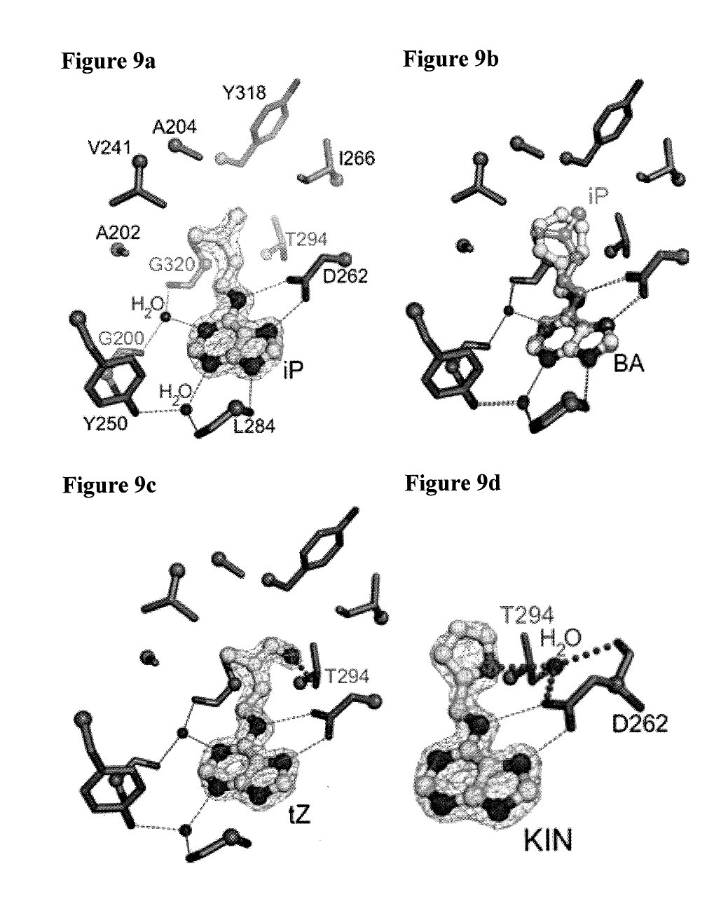

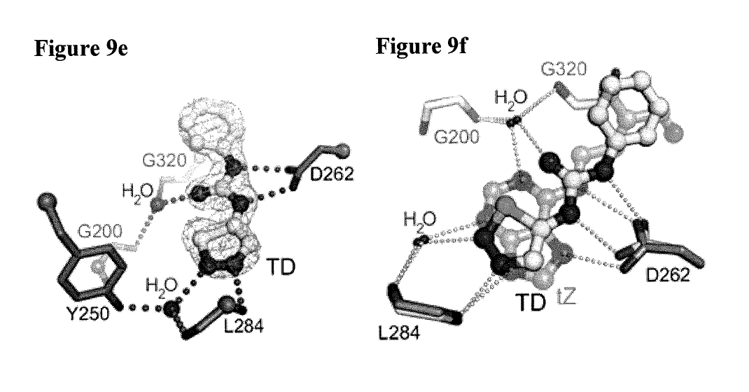

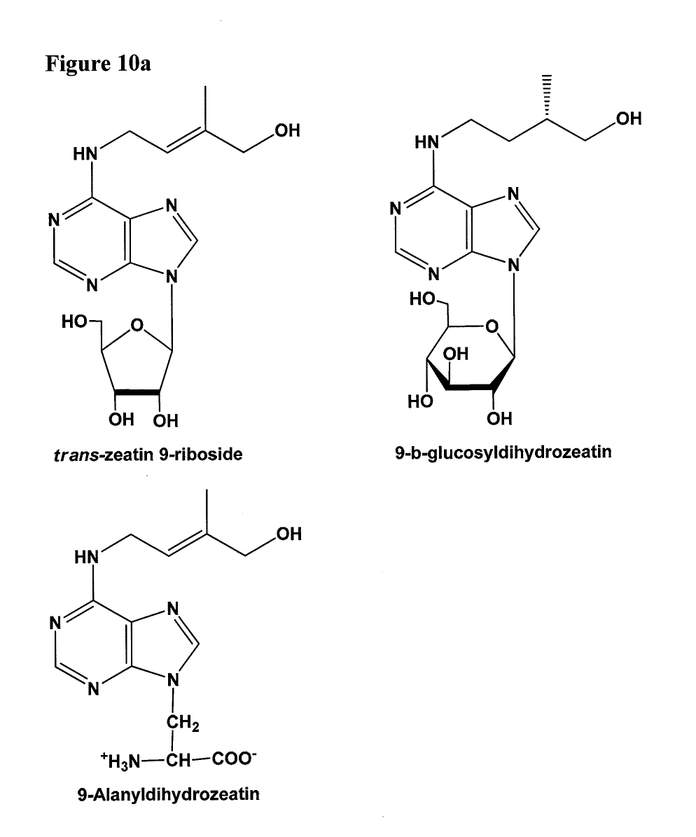

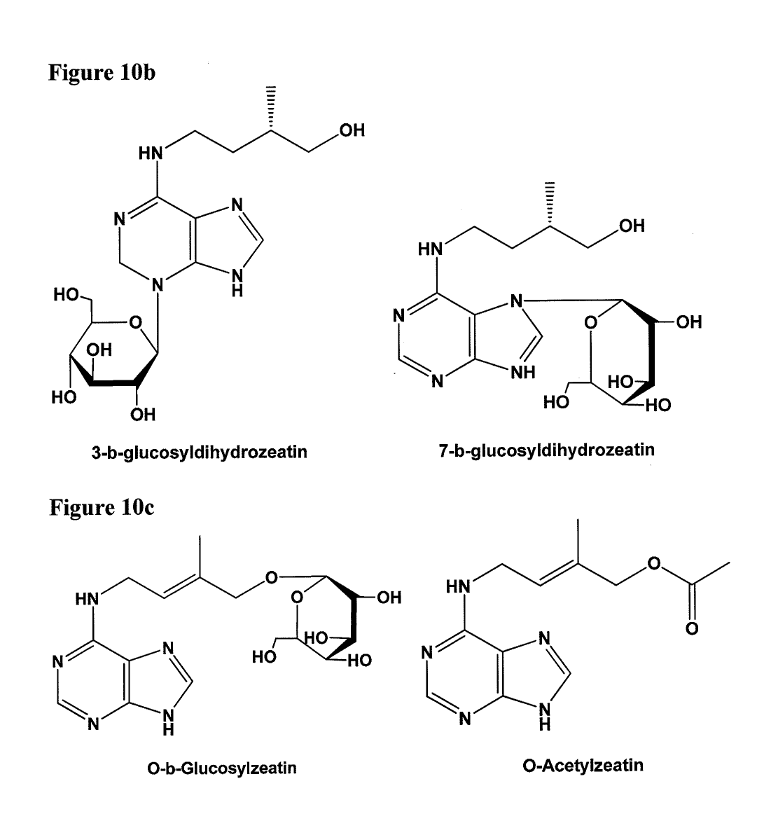

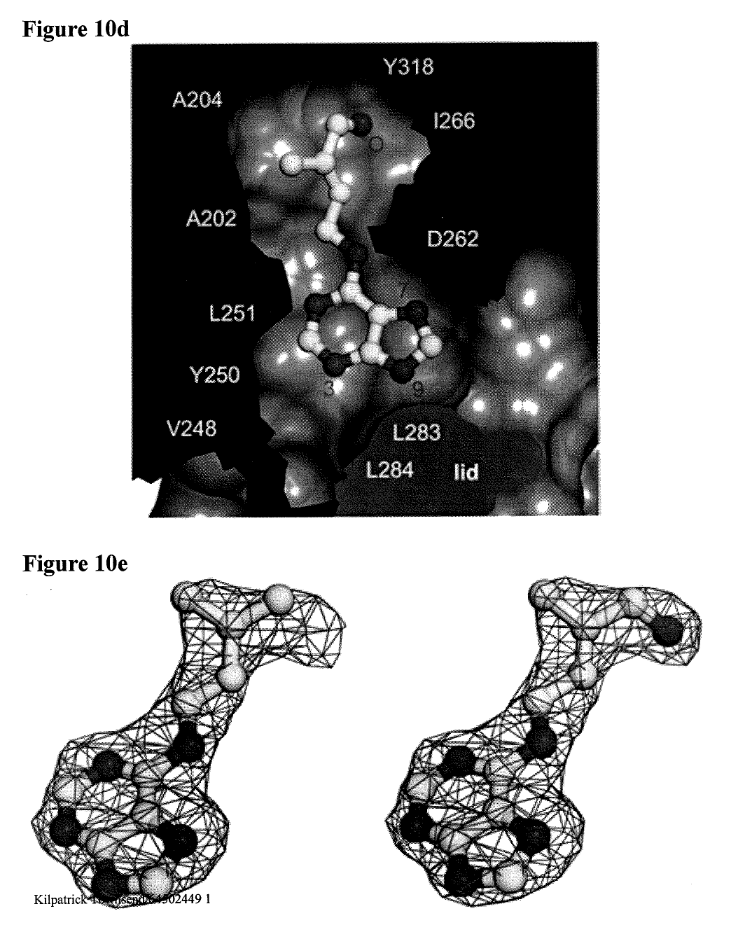

FIG. 3. Plant cytokinin receptors are structurally related to bacterial histidine kinase family 1 sensor domains. a, Structural superposition of the AHK4 sensor domain (C_{α} trace, in orange) and the extracellular domain from a senor histidine kinase from Bacillus subtilis (which is less than 15% sequence identical to AHK4, PDB-ID 2fos, in blue, r.m.s.d. is ˜2.1 Å comparing 181 corresponding C_{α} atoms). The N-terminal stalk helix and the membrane proximal PAS domain align well, while the cytokinin binding domain appears less conserved. A 3D homology search with the program DALI identified histidine kinase family 1 sensor domains that may share a common dimer interface with AHK4. b, Ribbon diagram of the AHK4 sensor domain, c, Bacillus subtilis HK sensor domain (PDB-ID: 3fos, DALI Z-score: 17.0), d, Sinorhizobium meliloti DctB (PDB-ID: 3e4p, DALI Z-score: 14.8), e, Shewanella oneidensis soHK1S-Z6 (PDB-ID: 3LIC, DALI Z-score 14.4). Colors are according to FIG. 1.

FIG. 4. The AHK4 sensor domain forms dimers. a, Residues in the dimer interface are highly conserved among known cytokinin receptors. The molecular surface is colored according to sequence conservation, ranging from dark orange for invariant residues to white for variable residues. b, Ribbon diagram of the dimer interface (in orange) in AHK4. c, Analytical gel filtration. Wild-type AHK4 eluates as a dimer, a double mutant in the dimer interface (Ile165A1a/Leu166A1a) as a monomer. Void (V₀) and total volume (V₁) are shown together with the elution volumes for molecular weight standards (A, conalbumin, MW 75,000, B, Ovalbumin, MW 43,000, C, carbonic anhydrase, MW 29,000, D, Ribonuclease A, MW 13,700, E, Aprotinin, MW 6,500). The estimated molecular weight for the dimer peak (black) and the monomer peak (red) are 66,000 and 39,000, respectively. The calculated monomer molecular weight is 30,700. d, Cytokinin induced β-galactosidase activity in AHK4 containing E. coli cells harboring mutations in the dimer interface. The mutations E186A anad R190A significantly inhibit cytokinin signaling.

FIG. 5. The AHK4 sensor domain forms constitutive dimers. a, Stereo close-up view of the dimerization interface in AHK4 adopting a pseudo 2-fold symmetry along the stalk helix. Molecules A and B of the dimer are colored in orange and blue, respectively. Interface residues are shown together with all polar interactions (dotted lines), mutated residues are highlighted in ball-and-stick representation. b, Structure based sequence alignment of the dimerization interface in different cytokinin receptors: AHK4, Arabidopsis histidine kinase 4, Arabidopsis thaliana (UniProt Acc. Q9SIT0), AHK2, Arabidopsis thaliana (Q9FKH3), AHK3, Arabidopsis thaliana (Q9C5U1), PtHK, Populus trichocarpa (B9GZP2), VvHK, Vitis vinifera (D7TAZ7), OsHK, Oryza sativa (Q6PX60), LjHK, Lotus japonicus (A0PEY0), ZmHK, Zea mays (Q2ACB8), PpHK, Physcomitrella patens (Q27PX4). Interface residues are highlighted, invariant residues are labeled with a *. The alignment includes a secondary structure assignment for AHK4 calculated with DSSP (Kabsch, W. & Sander, C., Biopolymers 22:2577-2637 (1983)). c, 280 nm absorbance trace of an analytical gel filtration. Wild-type AHK4 eluates as a dimer, as does the Asp262Arg mutant that likely interferes with hormone binding, and the gain-of-function mutation Leu333Phe in the transmitter helix. Void (V₀) and total volume (V₁) are shown together with the elution volumes for molecular weight standards (A, conalbumin, MW 75,000, B, Ovalbumin, MW 43,000, C, carbonic anhydrase, MW 29,000, D, Ribonuclease A, MW 13,700, E, Aprotinin, MW 6,500). The estimated molecular weight for the dimer peak is 66,000.

FIG. 6. Cytokinin binding induces a conformational change in AHK4. a, Ribbon diagram of an AHK4 monomer. The stalk helix is in orange, the lid is in magenta. Lid residue Arg282 interacts with G1u326 in the ‘transmitter’ helix (in cyan), that can move upon ligand-binding. Gain-of-function mutations in the ‘transmitter’ helix are highlighted. b, Close-up view on Va1329 and Leu333 in the membrane-proximal PAS domain. Selected parts of the hydrophobic core and the disulfide bridge are shown in bonds representation. c, Cytokinin induced β-galactosidase activity in AHK4 containing E. coli cells harboring mutations in the transmitter helix. The mutations V329F and L333F result in cytokinin signaling in the absence of tZ ligand.

FIG. 7. Hormone binding to the AHK4 cytokinin-binding pocket causes movements of the membrane helix TM2. Schematic diagrams of the constitutive AHK4 sensor domain dimer, colored according to FIG. 1. TM2 connects the sensor domain to the dimerization domain of the cytoplasmic histidine kinase module. a, Resting state. b, Activated state: Cytokinin binding to the membrane-distal PAS-like domain likely triggers movement of the ‘lid’ loop (in magenta), which in turn causes rearrangements in the transmitter helix (in cyan) and in the underlying membrane-proximal PAS domain. The data indicate that the disulfide bridge in AHK4 provides a pivot that translates these movements into changes of the relative x and/or y position of TM2 with respect to TM1, and with respect to TM2′ in the dimer partner. Gain-of-function mutations that alter or disrupt the dimer interface altogether (shown in yellow), mutations in the transmitter helix, or in TM2 itself (Miwa et al., Plant Cell Physiol. 48:1809-1814 (2007)), can all cause movement of TM2 in the absence of cytokinin ligand.

FIG. 8. Chemical structures of naturally occurring and synthetic cytokinins discussed in this study. Structures were drawn using chemtool (available at the website at ruby.chemie.uni-freiburg.de/˜martin/chemtool). a, N⁶-isopentenyl adenine (iP), b, N⁶-benzyladenine (BA), c, trans-Zeatin (tZ), d, dihydrozeatin (DZ), e, cis-Zeatin, f, Kinetin (KIN), g, diphenylurea (first synthetic cytokinin discovered), and h, the synthetic thiadiazuron (TD). Close-up of the cytokinin binding site occupied by DZ (in bonds representation). Interacting residues in the tail binding pocket are shown in blue.

FIG. 9. Structural plasticity in the PAS domain allows for the binding of diverse cytokinins. a, Close-up of the cytokinin binding site occupied by iP (in bonds representation, Fₒ-F_{c} omit electron difference density map is contoured at 4.5σ). Interacting residues in the tail binding pocket are shown in blue. b, Structural superpostion of the BA complex with a. c, The hydroxylated isopentenyl chain of tZ contacts Thr294 (in magenta). d, The tail binding pocket can accommodate the larger furfuryl group of KIN. A polar interaction (in magenta) with Thr294 is now mediated by a water molecule (in red). e, Structure of the TD complex with polar interactions included. The thiadiazol ring directly contacts the ‘lid’ loop (side-chain of Leu284 has been omitted for clarity). f, Structural superposition of the tZ (in gray) and TD (in yellow) complexes (r.m.s.d. is ˜0.15 Å between 266 corresponding C_{α} atoms). Interactions to Asp262 and Leu284 are included.

FIG. 10. Structural basis for the inactivation of natural cytokinins by enzymatic conjugation. Chemical structures of selected modified cytokinins, including a, N⁹ substituted cytokinins, b, N³ and N⁷ substituted cytokinins and c, O substituted cytokinins. d, Close-up of the AHK4 cytokinin binding pocket complexed with tZ. Shown is a molecular surface, the upper half of the binding pocket has been omitted for clarity. The structure suggests that N³, N⁷, and N⁹ substituted cytokinins should not bind to AHK4 efficiently, yet studies using an E. coli hormone binding assay indicated that trans-zeatin riboside (tZr), a N⁹ substituted cytokinin, binds to AHK4 with high affinity (Romanov et al., J. Exp. Bot. 57:4051-4058 (2006)). To address this discrepancy a complex of purified AHK4 in the presence of tZr was crystalized. Difference density in the ligand binding pocket e, indicates that E. coli iP had been displaced from the binding pocket, as additional density accounting for the hydroxyl group of tZ is evident (e, f). Difference density for the ribose portion of tZr (f), however, is not evident, indicating that the ligand underwent hydrolysis during purification. This hydrolysis likely occurred in the previous E. coli study, explaining the apparently discordant result.

### DETAILED DESCRIPTION OF THE INVENTION

### I. Introduction

Seven high resolution crystal structures of the AHK4 receptor were determined with aromatic and aliphatic adenine-based natural cytokinins, and with the urea-based thiadiazuron (a synthetic cytokinin, growth-regulator and defoliant). These structures revealed a highly adoptable hormone binding pocket in AHK4 and provide design principles for novel synthetic cytokinins and receptor antagonists.

The results disclosed herein rationalize previously established genetic studies, including receptor loss- and gain-of-function alleles and mutants. Using an in vivo reporter system for AHK4 receptor function, structure-based mutations revealed that cytokinin binding to the hormone binding domain triggers conformational changes in the sensor domain that ultimately lead to movements of the membrane helices. The membrane helices connect the sensor domain to the cytoplasmic HK domain, so that their movement relays a signal to activate the receptor.

Taken together the present results show the structural basis for ligand specificity, receptor activation, and the evolutionary origin of plant cytokinin receptors. The results allow for rational design of new synthetic cytokinins and receptor antagonists. Synthetic cytokinins can be used as growth regulators and defoliants. Depending on the target, receptor antagonists can be used to delay leaf senescence, promote seed germination, increase hypocotyl (shoot) and root length, etc.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989); Raven et al. PLANT BIOLOGY (7^{th} ed. 2004). Any methods, devices and materials similar or equivalent to those described herein can be used in the practice of this invention. The following definitions are provided to facilitate understanding of certain terms used frequently herein and are not meant to limit the scope of the present disclosure.

A cytokinin receptor refers to a receptor exemplified by the Arabidopsis cytokinin receptors AHK2, AHK3, and AHK4 (see Ferreira & Kieber (2005) Curr. Opin. Plant Biol. 8:528). Cytokinin receptor domains include the cytokinin binding CHASE (cyclases/histidine kinases associated sensory extracellular) domain, the histidine kinase (HK) domain, the receiver domain, the dimerization domain, and 2 transmembrane domains.

Cytokinins include both naturally occurring and synthetic plant hormones that bind to cytokinin receptors. Naturally occurring cytokinins are adenine based, and included iP, BA, tZ, and KIN. Synthetic cytokinins include TD, and other urea based compounds as known in the art.

A “cytokinin analog” or “cytokinin mimetic” is a compound that has a similar structural interaction with cytokinin receptor as a natural or synthetic cytokinin (e.g., the interaction between AHK4 and iP). Cytokinin mimetics include iP analogs and mimetics, KIN analogs and mimetics, BR analogs and mimetics, tZ analogs and mimetics, TD analogs and mimetics, etc. In some cases, the cytokinin mimetic also has cytokinin activity and activates cytokinin receptor signaling. Cytokinin mimetics can be adenine or urea-based, aromatic or aliphatic, and include purine derivatives andside chain analogs (e.g., C-6 side chain analogs). Cytokinin mimetics also include compounds that are designed considering the cytokinin receptor structure provided herein, e.g., to have a similar interaction with cytokinin receptor as a cytokinin, as shown in FIG. 2, and described in Tables 1 and 2.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes, biotin, digoxigenin, or haptens and proteins or other entities which can be made detectable, e.g., by incorporating a radiolabel into a peptide or antibody specifically reactive with a target peptide. Any method known in the art for conjugating a compound or protein to the label may be employed, e.g., using methods described in Hermanson, Bioconjugate Techniques 1996, Academic Press, Inc., San Diego.

A “labeled” or “tagged” molecule (e.g., compound, modulator, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

“Nucleic acid” refers to deoxyribonucleotides or ribonucleotides and polymers thereof in either single- or double-stranded form, and complements thereof The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman, Add. APL. Math. 2:482 (1981), by the homology alignment algorithm of Needle man and Wunsch, J. Mol. Biol. 48:443 (1970), by the search for similarity method of Pearson and Lipman, Proc. Natl. Acad. Sci. U.S.A. 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wiss.), or by inspection.

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the preferred algorithms can account for gaps and the like. Preferably, identity exists over a region comprising a ligand binding site or interaction domain, or a sequence that is at least about 25 amino acids or nucleotides in length, or over a region that is 50-100 amino acids or nucleotides in length.

The term “recombinant” when used with reference, e.g., to an organism, cell, nucleic acid, protein, or vector, indicates that the organism, cell, nucleic acid, protein or vector has been modified by the introduction of a heterologous nucleic acid or protein or the alteration of a native nucleic acid or protein, or that the cell is derived from a cell so modified. Thus, for example, recombinant cells and organisms express genes that are not found within the native (non-recombinant) form of the cell or express native genes that are otherwise abnormally expressed, under expressed or not expressed at all.

A polynucleotide or polypeptide is “heterologous to” a second polynucleotide or polypeptide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants.

An “expression cassette” refers to a nucleic acid construct, which when introduced into a host cell, results in transcription and/or translation of a RNA or polypeptide, respectively. Antisense or sense constructs that are not or cannot be translated are included by this definition.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, bryophytes, and multicellular algae. It includes plants of a variety of ploidy levels, including aneuploid, polyploid, diploid, haploid and hemizygous.

The term “specifically bind” refers to a compound (e.g., cytokinin receptor-binding compound) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity.

The term “compete”, as used regarding a cytokinin receptor ligand, modulator, or interacting protein, means that a first compound competes for binding to cytokinin receptor with a second compound, where binding of the first compound to its site on cytokinin receptor is detectably decreased in the presence of the second compound compared to the binding of the first compound in the absence of the second compound. The alternative, where the binding of the second compound to its site on cytokinin receptor is also detectably decreased in the presence of the first compound, can, but need not be the case. That is, a first compound can inhibit the binding of a second compound to its site without that second compound inhibiting the binding of the first compound to its respective site. However, where each compound detectably inhibits the binding of the other to cytokinin receptor, whether to the same, greater, or lesser extent, the compounds are said to “cross-compete” with each other for binding of their respective site(s).

The term “modulator” includes inhibitors and activators. Inhibitors are agents that, e.g., inhibit expression or bind to, partially or totally block stimulation or down regulate the activity of the described target protein, e.g., a cytokinin receptor such as AHK4. Activators are agents that, e.g., induce or activate the expression of a described target protein or bind to, stimulate, or up regulate the activity of described target protein, e.g., cytokinin receptor. Modulators include naturally occurring and synthetic ligands, antagonists and agonists (e.g., small chemical molecules, hormones, antibodies, etc. that affect target activity). Assays for inhibitors and activators include, e.g., applying candidate modulator compounds to cells expressing the described target protein (e.g., cytokinin receptor expressing cells) and then determining the functional effects on the described target protein activity. Samples or assays comprising described target protein that are treated with a potential activator, inhibitor, or modulator are compared to control samples without the inhibitor, activator, or modulator to examine the extent of effect. Control samples (untreated with modulators) can be assigned a relative activity value of 100%.

The terms “agonist,” “activator,” “inducer” and like terms refer to molecules that increase activity or expression as compared to a control. Agonists are agents that, e.g., bind to, stimulate, increase, activate, enhance activation, sensitize or upregulate the activity of the target. The expression or activity can be increased 10%, 20%, 30%, 40%, 50%, 60%, 70%, 80%, 90% 100% or more than that of a control (i.e., 110%, 120%, etc.). In certain instances, the activation is 1.5-fold, 2-fold, 3-fold, 4-fold, 5-fold, 10-fold, or more in comparison to a control.

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound (e.g., candidate cytokinin receptor or AHK4 modulator), and compared to samples from known conditions, e.g., in the absence of the test compound (negative control), or in the presence of a known compound (positive control, e.g. iP or other known cytokinin receptor modulator). A control can also represent an average value gathered from a number of tests or results. One of skill in the art will recognize that controls can be designed for assessment of any number of parameters. Controls can be designed for in vitro applications, e.g., for comparison to the binding activity and location of various candidate cytokinin receptor modulators. Controls can also be designed for in situ or in vivo applications, e.g., for comparison to the effect of candidate cytokinin receptor modulators on a cytokinin receptor-expressing plant or plant part. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As described below in detail, we have crystallized the AHK4 cytokinin receptor sensor domain as associated with various natural and synthetic ligands (see, e.g., Tables 1 and 2). Several parameters can be used to uniquely describe the symmetry and geometrical characteristics of a crystal. These include the space group (symmetry), the three unit cell axial lengths “a”, “b”, and “c”, and the three unit cell interaxial angles “α”, “β”, and “γ” (geometry). “Unit cell axial length” and “unit cell interaxial angle” are terms of art that refer to the three-dimensional geometrical characteristics of the unit cell, in essence its length, width, and height, and whether the building block is a perpendicular or oblique parallelepiped. The unit cell axial lengths and interaxial angles can vary by as much as ±10% without substantively altering the arrangement of the molecules within the unit cell. Thus, reference to each of the unit cell axial lengths and interaxial angles as being “about” a particular value is to be understood to mean that any combination of these unit cell axial lengths and interaxial angles can vary by as much as ±10% from the stated values.

### III. Methods for Protein Expression

Cytokinin receptors and receptor domains (e.g., sensor, transmembrane, PAS, dimerization, ligand binding, or cytoplasmic domains), and the like can be recombinantly expressed according to methods known in the art (see, e.g., Mus-Vetaux, Heterologous Expression of Membrane Proteins (2009); Glorioso et al. Expression of Heterologous Genes in Eukaryotic Systems, Methods in Enzymology Vol.306 (1999)).

The sequence and domains for cytokinin receptors are publically available at the NCBI website (ncbi.nlm.nih.org) for several plant species. For example, the Arabidopsis Genbank accession numbers for AHK2, AHK3, and AHK4 are NP_{—}568532.1, NP_{—}564276.1, and AEC05507.1 respectively (AHK4 is shown as SEQ ID NO:1). One of skill will understand that species homologs can be optimally aligned so that conserved residues can be located on the respective cytokinin receptor proteins.

Provided herein are recombinant expression cassettes comprising a promoter sequence operably linked to a nucleic acid sequence encoding a desired polypeptide sequence (e.g., cytokinin receptors, cytokinin receptor variants and species homologs, a cytokinin receptor domain, etc.). In some embodiments, the cytokinin receptor domain is a sensor domain, a ligand binding domain, a transmembrane domain, a dimer interaction domain, or a PAS domain.

To use isolated sequences in the above techniques, recombinant DNA vectors suitable for transformation of cells can be prepared. Techniques for transforming a wide variety of higher plant species are well known and described in the technical and scientific literature, e.g., Weising et al., Ann. Rev. Genet. 22:421-477 (1988). Methods for expression in insect cells are described in more detail in the examples. Any cell type can be used for overexpression and protein production, as will be familiar to one of skill in the art, and kits for protein expression and purification are commercially available (e.g., from Invitrogen). A DNA sequence coding for the desired polypeptide, for example a cDNA sequence encoding a full length protein, can be combined with transcriptional and translational initiation regulatory sequences which will direct the transcription of the sequence from the gene in the intended cell. In the context of the present invention, protein expression for the purpose of in situ or in vivo functional studies is typically carried out in plant cells, plant tissues, or whole plants (transgenic plants).

For example, a plant promoter can be employed which will direct expression of the gene in all tissues of a regenerated plant. Such promoters are referred to herein as “constitutive” promoters and are active under most environmental conditions and states of development or cell differentiation. Alternatively, the plant promoter can direct expression of the polynucleotide of the invention in a specific tissue (tissue-specific promoters, organ-specific promoters) or specific environmental condition (inducible promoters).

A polyadenylation region at the 3′-end of the coding region can be included. The polyadenylation region can be derived from the natural gene, from a variety of other plant genes, or from T-DNA.

The vector comprising the sequences (e.g., promoters or coding regions) from genes of the invention will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Coding sequences, e.g., nucleic acid sequences that encode the cytokinin receptor protein or domain, can expressed recombinantly in plant cells. A variety of different expression constructs, such as expression cassettes and vectors suitable for transformation of plant cells can be prepared. A DNA sequence coding for a polypeptide described in the present invention, e.g., a cDNA sequence encoding cytokinin receptor, or a cytokinin receptor domain, can be combined with cis-acting (promoter and enhancer) transcriptional regulatory sequences to direct the timing, tissue type and levels of transcription in the intended tissues of the transformed plant. Translational control elements can also be used.

The invention provides a nucleic acid encoding a cytokinin receptor polypeptide operably linked to a promoter which is capable of driving the transcription of the coding sequence in plants. The promoter can be, e.g., derived from plant or viral sources. The promoter can be, e.g., constitutively active, inducible, or tissue specific. In construction of recombinant expression cassettes, vectors, transgenics, of the invention, different promoters can be chosen and employed to differentially direct gene expression, e.g., in some or all tissues of a plant.

Further provided are methods of generating transgenic plants that express recombinant cytokinin receptor (or a domain thereof, or another desired protein). Appropriate expression cassettes can be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using biolistics, e.g., DNA particle bombardment.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Biolistic transformation techniques are described in Klein et al. Nature 327:70-73 (1987).

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983) and Gene Transfer to Plants, Potrykus, ed. (Springer-Verlag, Berlin 1995).

Transformed plant cells which are derived by any of the above transformation techniques can be cultured to regenerate a whole plant which possesses a desired phenotype.

Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof Such regeneration techniques are described generally in Klee et al., Ann. Rev, of Plant Phys. 38:467-486 (1987).

The above techniques can be used to produce transgenic plants in any plant species, including species from the genera Anacardium, Arachis, Asparagus, Atropa, Avena, Brassica, Chlamydomonas, Chlorella, Citrus, Citrullus, Capsicum, Carthamus, Cocos, Coffea, Cucumis, Cucurbita, Cyrtomium, Daucus, Elaeis, Fragaria, Glycine, Gossypium, Helianthus, Heterocallis, Hordeurn, Hyoscyamus, Lactuca, Laminaria, Linum, Lolium, Lupinus, Lycopersicon, Macrocystis, Malus, Manihot, Majorana, Medicago, Nereocystis, Nicotiana, Olea, Oryza, Osmunda, Panieum, Pannesetum, Persea, Phaseolus, Pistachia, Pisum, Pyrus, Polypodium, Prunus, Pteridium, Raphanus, Ricinus, Secale, Senecio, Sinapis, Solanum, Sorghum, Theobromus, Trigonella, Triticum, Vicia, Vitis, Vigna, and Zea.

### IV. Methods of Identifying a Cytokinin Receptor Modulator

### A. Cytokinin Receptor Activity Assays

Cytokinin receptor activities include binding to natural and synthetic cytokinins (e.g. iP, BA, KIN, tZ, TD, DZ, BAP), and cytokinin receptor antagonists, such as PI-55 and the compounds disclosed in Nisler et al. (2010) Phytochemistry 71:823. Cytokinin receptor modulators can also bind to cytokinin receptor, e.g., to interfere with ligand binding or dimerization (antagonist), or to mimic or improve ligand binding (agonist).

The binding affinity of a compound, e.g., a candidate cytokinin receptor modulator, can be defined in terms of the comparative dissociation constants (Kd) of the compound for target (e.g., AHK4), as compared to the dissociation constant with respect to the compound and other materials in the environment or unrelated molecules in general. Typically, the Kd for the compound with respect to the unrelated material will be at least 2-fold, 3-fold, 4-fold, 5-fold, 10-fold, 20-fold, 50-fold, 100-fold, 200-fold or higher than Kd with respect to the target.

The desired affinity for an compound, e.g., high (pM to low nM), medium (low nM to 100nM), or low (about 100nM or higher), can differ depending upon the cytokinin receptor binding site and the targeted activity. Compounds having different affinities can be used for different applications.

A compound will typically bind with a Kd of less than about 1000 nM, e.g., less than 250, 100, 50, 20 or lower nM. In some embodiments, the Kd of the compound is less than 15, 10, 5, or 1 nM. The value of the dissociation constant (Kd) can be determined by well-known methods, and can be computed even for complex mixtures by methods as disclosed, e.g., in Caceci et al., Byte (1984) 9:340-362; and as reviewed in Ernst et al. Determination of Equilibrium Dissociation Constants, Therapeutic Monoclonal Antibodies (Wiley & Sons ed. 2009).

The Kd, Kon, and Koff can also be determined using surface plasmon resonance (SPR), e.g., as measured by using a Biacore T100 system. SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding.

Binding affinity can also be determined by anchoring a biotinylated interactant to a streptaviden (SA) sensor chip. The other interactant is then contacted with the chip and detected, e.g., as described in Abdessamad et al. (2002) Nuc. Acids Res. 30:e45.

Cytokinin receptor activities also include initiation of the cytokinin signaling pathway and upregulation ARR5 expression. Assays for detection of cytokinin receptor signal transduction are described, e.g., in Coba de la Pena et al. (2008) Plant Signal Behav. 13:791.

Methods for detecting cytokinin receptor activities, such as regulation of growth and differentiation (e.g., induction of leaf senescence, slowing of seed germination, restraining hypocotyl (shoot) and root length) can include the steps of exposing a cytokinin receptor expressing plant with a cytokinin receptor agonist and detecting an increase in cytokinin receptor activity (e.g. cytokinin receptor signaling) as compared to a standard control. An appropriate standard control can be selected by one of skill in the art, e.g., a plant that does not express cytokinin receptor, a plant that is not exposed to a cytokinin receptor agonist, or a plant that is exposed to a cytokinin receptor antagonist. Such methods are described, e.g., in Nisler et al. (2010) Phytochemistry 71:823. Cytokinin receptor activity can be measured using classical cytokinin assays, such as tobacco callus growth, Amaranthus betacyanin synthesis, and excised leaf senescence (Holub et al. (1998) Plant Growth Regul. 26:109).

In some embodiments, the invention provides methods of identifying a cytokinin receptor modulator comprising contacting a candidate compound and cytokinin receptor, and detecting cytokinin receptor activity, wherein a change in cytokinin receptor activity in the presence of the candidate compared to a standard control indicates that the candidate compound is a cytokinin receptor modulator. In some embodiments, the cytokinin receptor is expressed in a plant, and the contacting step involves contacting the candidate compound with the plant. In some embodiments, the cytokinin receptor modulator is a cytokinin receptor inhibitor, and in some embodiments, the cytokinin receptor modulator is a cytokinin receptor agonist.

In some embodiments, the standard control lacks the candidate compound. In some embodiments, e.g., for determining whether the candidate compound is a cytokinin receptor agonist, the standard control is iP, or another known cytokinin. In some embodiments, e.g., for determining whether the candidate compound is a cytokinin receptor antagonist, the method further includes a step of exposing the cytokinin receptor to an agonist, and determining the ability of the candidate compound to interfere with cytokinin receptor signaling. In some embodiments, the inhibitory activity of the potential cytokinin receptor antagonist is compared to that of PI-55.

The presently provided structural data allows one of skill to more accurately design and/or identify potential cytokinin receptor modulators, e.g., based on known modulators, the structural elements of the ligand-binding site, or the structural elements of dimer interaction site.

### B. Cytokinin Receptor Variants and Modulators

Provided herein are cytokinin receptor variants and modulators that can be used for comparison, e.g., as controls, in the screening methods described herein. For example, the activity of a candidate cytokinin receptor modulator can be compared to that of a known cytokinin receptor modulator. The activity of a candidate cytokinin receptor modulator can also be compared to the activity of a cytokinin receptor variant, e.g., a gain-of-function mutant (Tirichine et al. (2007) Science 315:104) or loss-of-function mutant (Chang et al. (1993) Science 262:539).

Cytokinin receptor variants include loss-of-function mutants Thr278Ile, Asp262Arg, and mutations in residues 280-284, while gain-of-function mutations include Leu333Phe and Va1329Phe.

Compounds with cytokinin receptor agonist activity include, but are not limited to, the natural and synthetic cytokinins disclosed herein (iP, BA, TD, tZ, KIN, and DZ). The activity of a candidate cytokinin receptor modulator can be compared with these cytokinin receptor agonists to determine if the candidate modulator is also an agonist. Cytokinin receptor antagonists include PI-55 and the 6-benzylaminopurine derivatives disclosed in Nisler et al. (2010), which can be used for comparison, e.g., to determine if a candidate modulator is an antagonist.

### C. Rational Design of Cytokinin Receptor Modulators

Hormones, hormone mimetics, and other modulating compounds with cytokinin receptor regulating activity can be identified using structure coordinates of the cytokinin receptor sensor domain, or other cytokinin receptor domains, as disclosed herein. Such methods of screening can comprise: (a) generating structure coordinates of a three-dimensional structure of a test substance; and (b) superimposing the structure coordinates of (a) onto all or some of the structure coordinates of cytokinin receptor in the same coordinate system so as to evaluate their state of fitting. Specifically, such a method involves fitting the structure coordinates of cytokinin receptor to structure coordinates representing a three-dimensional structure of any test substance on a computer, expressing their state of fitting numerically using, for example, empirical scoring functions as indices, and then evaluating the binding ability of the test substance to cytokinin receptor.

The structure coordinates of cytokinin receptor are used, the shape of cytokinin receptor binding site or interaction site is assigned, and then a compound that can bind to the site can be subjected to computer screening using commercial package software such as DOCK (Ewing et al., J. COMP. AIDED MOL. DES. 15:411-428 (2001)), AutoDock (Morris et al., J. COMPUTATIONAL CHEM. 19:1639-1662 (1998)), Ludi, or LigandFit. For example, amino acid residues and domains in cytokinin receptor that can interact with cytokinin ligand are shown, e.g., in FIGS. 1, 2, and 9. Thus, it becomes possible to conduct computer screening using such sites as an aid.

The step of superimposing structure coordinates of a test substance onto all or some of the structure coordinates of cytokinin receptor in the same coordinate system so as to evaluate their state of fitting can also be carried out with the above commercial software. Any appropriate modeling software can be used, as long as it makes a simulation of the docking procedure of a ligand or other modulator to a protein possible on a computer. For example, software programs such as DOCK, FlexX (Tripos, Inc.), LigandFit (Accelrys Inc.), or Ludi (Accelrys Inc.) can be used.

In some embodiments, an initial step is positioning of a virtual spherical body referred to as a sphere, using a SPHGEN program, near a position to which a candidate cytokinin receptor modulator (agonist or antagonist) is likely to bind. This sphere functions as an anchor for docking of the modulator. In addition, sites at which spheres are generated can be limited to specific pockets or specific clefts, or spheres can be generated at a plurality of sites.

Next, grids are generated at a portion and the periphery of the desired cytokinin receptor position using a GRID program, so as to express an electronic and steric environment for receptor residues within an assigned range as a scalar value on each grid. In addition, the force field of AMBER (Pearlman, et al., COMP. PHYS. COMMUN. 91:1-41 (1995)) or the like is utilized to calculate each grid value. Furthermore, depending on the shape, adjustment can also be made by altering grid information so as to express docking of a compound in a more realistic form.

Next, a search can be conducted on a compound database. Using the DOCK program, a compound that is takes a three-dimensional conformation so as not to repel steric elements or electronic elements on the grids is searched for. The three dimensional conformation of the docked compound is optimized by a conformation-generating function integrated in the DOCK program. Whether or not appropriate docking is finally conducted can be further determined based on empirical judgment, e.g., using scores at the time of docking, visual observation, and in situ screening. In this manner, a series of selected compound groups judged to be able to appropriately conduct docking can be considered as substances likely to modulate cytokinin receptor activity (agonist or antagonist) at a certain probability.

The above method promotes more efficient, rational development of cytokinin receptor modulators. Specifically, predicting the arrangement of structure coordinates that fit the properties and shapes of the interaction sites of the cytokinin receptor-ligand complex, or the dimerized cytokinin receptor complex, and the selection by calculation of a compound having a structure capable of agreeing with the putative structure coordinates, make it possible to efficiently select an activity-controlling substance specific to cytokinin receptor from among many compounds.

Likely modulator compounds obtained from the modeling methods can then be validated using any of the screening methods described above, e.g., by contacting the likely modulator compound with a plant expressing cytokinin receptor, and determining the effect of the compound on the plant.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

### V. EXAMPLES

### A. Methods Summary

The AHK4 sensor domain was expressed as a SUMO fusion protein in E. coli origami cells and purified by tandem affinity chromatography and gel filtration. Purified samples were concentrated to 12 mg/mL in 20 mM Hepes (pH 7.5), 100 mM NaCl, 1 mM EDTA and crystallised using the vapour diffusion method with the reservoir solution containing 16-19% (v/v) PEG 3350, 0.2 M Na malonate (pH 5.0). The structure was solved by single isomorphous replacement using an iodine derivative and refined to 1.7 Å resolution with an R factor 17.6% of and an R_{free} of 20.1%. Complexes with different cytokinins were obtained by displacing the endogenously bound iP, and crystallised in conditions similar to the wild-type protein. Functional assays were performed as previously described (Mizuno & Yamashino, Methods in Enzymology 471:335-356 (2010)).

Protein Expression and Purification. A synthetic gene comprising the sensor domain of AHK4 (residues 126-395, numbering in accordance with the short splice-variant of AHK4) was codon-optimised for expression in E. coli (Geneart, Regensburg, Germany). The Ncol/Notl digested synthetic fragment was inserted into a modified pET21d (Novagen) plasmid providing N-terminal 7xHis and Strep II affinity tags and expressed as a SUMO fusion protein in E. coli Origami (DE3) cells (Novagen). Cells grown to an OD₆₀₀ₙₘ of 0.8 were induced with 0.3 mM isopropyl-β-D-thiogalactopyranoside in Terrific Broth at 16° C. for 22 h. Cells were collected by centrifugation at 4,500 xg for 30 min, washed in PBS buffer, incubated with 100 mg lysozyme for 1 h at room temperature and snap-frozen in liquid nitrogen. For protein purification cells were thawn in buffer A (25 mM Tris, pH 7.6, 500 mM NaCl, 15 mM Imidazole and, if applicable, 0.1 mM of the respective cytokinin), lysed by sonication, and centrifuged at 75,000 xg for 90 min. The supernatant was incubated in batch with 5 mL HIS-Select Cobalt Affinity Gel (Sigma), washed with buffer A and eluted in buffer A supplemented with 200 mM imidazole. The fusion protein was dialysed against buffer B (25 mM Tris, pH 8.0, 300 mM NaCl, 1 mM EDTA) and then loaded onto a 1 mL Strep-Tactin Superflow high capacity cartridge (IBA, Gottingen, Germany). The column was washed with 8 mL of buffer B and the fusion protein was eluted against a step gradient of buffer B supplemented with 2 mM D-Destiobiotin. The N-terminal SUMO tag was cleaved by adding the catalytic domain of ScUlpl (Reverter & Lima, Methods Mol. Biol. 497:225-239 (2009)) (residues 403-621) in 1:50 ratio for 16 h at 4° C. during dialysis against buffer B. Next, AHK4 was separated from the Strep II-tagged SUMO protease and SUMO fusion tag by a second Strep-Tactin affinity step and by gel filtration on a Superdex 200 HR10/30 column (GE Healthcare), equilibrated in buffer B. Dimeric peak fractions were dialysed against 20 mM Hepes (pH 7.5), 100 mM NaCl, 1 mM EDTA (and, if applicable, 0.5 mM of the respective cytokinin), and concentrated to 12 mg/mL for crystallization. Site specific mutations were introduced by PCR and purified as described above. Depending on the preparation, about 25-100 μg of purified AHK4 protein could be obtained from 1 L of bacterial cell culture.

Crystalization and Data Collection. Hexagonal crystals of AHK4 were grown at room temperature from hanging drops composed of 1.5 μL of protein solution and 1.5 μL of crystalization buffer (16-19% (v/v) PEG 3350, 0.2 M Na malonate, pH 5.0) suspended over 1.0 mL of the latter as reservoir solution in several rounds of microseeding. For structure solution crystals were derivatized and cryo-protected by serial transfer into reservoir solution containing a final concentration of 25% (v/v) ethylene glycol and 0.5 M KI, and snap-frozen in liquid N₂.

Single-wavelength anomalous diffraction (SAD) data were collected on a Rigaku MicroMax rotating anode equipped with a copper filament, osmic mirrors and an R-AXIS IV++ detector. Subsequently, an isomorphous native dataset was collected on a crystal that originated from the same crystalization drop, with 0.5 M LiCl replacing the KI in the cryo-solution (Table 1). Ligand complexes crystalized under similar conditions using microseeding protocols. High resolution data sets were collected at beam-lines BL 8.2.1 and 8.2.2 of the Advanced Light Source (ALS) in Berkeley, USA. Data processing and scaling was done with XDS (Kabsch, J. Appl. Crystallogr. 26:795-800 (1993)) (version: May 2010) (Tables 1 and 2).

Structure Solution and Refinement. Native and derivative data sets were scaled with XPREP (Bruker AXS, Madison, Wiss.) for SIRAS (single isomorphous replacement with anomalous scattering) phasing. The program SHELXD (Sheldrick, Acta Crystallogr., A, Found. Crystallogr. 64:112-122 (2008)) was used to locate 40 iodine sites. 10 consistent sites were input in the program SHARP (Bricogne et al., Acta Crystallogr. D Biol. Crystallogr. 59:2023-2030 (2003)) for phasing and identification of 9 additional sites at 2.8 Å resolution. Refined heavy-atom sites and phases were input into PHENIX.AUTOBUILD (Terwilliger et al., Acta Crystallogr. D Biol. Crystallogr. 64:61-69 (2008)) for detection of 2-fold NCS symmetry, density modification and phase extension to 2.35 Å and later on to 1.7 Å resolution. The structure was completed in alternating cycles of model correction in COOT (Emsley & Cowtan, Acta Crystallogr. D Biol. Crystallogr. 60:2126-2132 (2004)) and restrained TLS refinement in PHENIX.REFINE (Afonine et al., CCP4 Newsl. contribution 8 (2005)) and REFMAC5 (Winn, et al., Acta Crystallogr. D Biol. Crystallogr. 57:122-133 (2001)). Cytokinin coordinate files and corresponding geometry restraints were generated with PRODRG2 (Schüttelkopf & van Aalten, Acta Crystallogr. D Biol. Crystallogr. 60:1355-1363 (2004)) and PHENIX.ELBOW (Moriarty et al., Acta Crystallogr. D Biol. Crystallogr. 65:1074-1080 (2009)), respectively. Phasing and refinement statistics are summarized in Tables 1 and 2. Loop residues 365-368 and 392-395 appear disordered in most structures. Analysis with MOLPROBITY (Davis et al., Nucleic Acids Res. 35:W375-383 (2007)) indicated excellent stereochemistry for all refined models. Structural visualisation was done with POVScript+ (Fenn et al., J. Appl. Crystallogr. 36:944-947 (2003)) and POV-Ray (available from the website at povray.org).

Analytical size-exclusion chromatography was performed using a Superdex 75 HR 10/30 column (GE Healthcare) pre-equilibrated in 25 mM Hepes (pH 7.0), 150 mM NaCl, 1 mM EDTA. 100 μL of sample (3.5 mg/mL) was loaded onto the column and elution at 0.7 mL/min was monitored by ultraviolet absorbance at 280 nm.

Semi-quantitative AHK4 histidine kinase assay in E. coli was performed as described (Mizuno & Yamashino, Methods in Enzymology 471:335-356 (2010)). The method relies on the functional expression of AHK4 in E. coli strain KMI001 lacking the sensor histidine kinase RcsC (rcsC::Kmr, wza::lacZ Δcps). In this way, AHK4 receptor function can be read out as β-galactosidase activity. Specifically, wild-type and mutant forms of AHK4 in plasmid pCold IV (Takara Bio, Otsu, Shiga, Japan) were transformed into chemically competent KMI001 cells and plated on LB-agar plates. The next day, single colonies were transferred to glucose-LB-agar plates containing X-Gal (40 μg/mL) and either 0 or 1 μM of tZ. Plates were incubated for 48 h at 25° C.

### B. Example 1

### Structure of AHK4 Sensor Domain with N⁶-isopentenyl adenine

To determine how Arabidopsis cytokinin receptors recognize cytokinins, we expressed the sensor domain of AHK4 (residues 126-395) and determined its crystal structure to 1.7 Å resolution (Table 1). The N-terminus of AHK4 folds into a long stalk helix followed by two PAS-like domains (Ponting & Aravind, Curr. Biol. 7:R674-677 (1997)) which are connected by a helical linker (FIG. 1a). The last β-strand of the membrane-proximal PAS domain is covalently linked to the N-terminus of the stalk helix by a disulfide bridge, bringing the membrane helices into close proximity (FIG. 1a). AHK4 recognizes cytokinins with its membrane-distal PAS domain as indicated by the presence of N⁶-isopentenyl adenine (iP) in our structure (FIG. 1a,b). iP is a natural cytokinin also present in bacteria, where it is generated during tRNA degradation (Caillet & Droogmans, J. Bacteriol. 170:4147-4152 (1988); Gray et al., Plant Physiol. 110:431-438 (1996)). E. coli iP binds to AHK4 with nanomolar affinity (Romanov et al., J. Exp. Bot. 57:4051-4058 (2006)) and thus co-purified. The ligand binding pocket is occupied by both the adenine portion of iP and by its isopentenyl tail, which is deeply inserted into the cavity (FIG. 1b). The lower half of the binding site is formed by the central β-sheet of the PAS domain and is lined by small hydrophobic residues (FIG. 1b, FIG. 2). Introducing bulkier amino-acids in this area inactivates AHK4, as judged by a functional assay in E. coli. (Mizuno & Yamashino, Methods in Enzymology 471:335-356 (2010)) (FIG. 1c). Two β-strands form the upper half of the pocket and contribute additional hydrophobic contacts (FIG. 1b). Hydrogen bonds are established between Asp262 and the adenine ring and these interactions appear critical for receptor function (FIG. 1b,c). Other polar interactions are mediated by water molecules, which in turn contact main-chain atoms (FIG. 1b). Mutation of Thr278 to Ile (the wooden leg allele) likely restricts the overall size of the binding pocket, and thus the present structure rationalizes the associated loss-of-function phenotype (FIG. 1b,c, FIG. 2).

### C. Example 2

### AHK4 Dimerization

We next investigated how cytokinin binding to the sensor domain triggers receptor activation. Previous analyses had grouped AHK4 into the CHASE (cyclases/histidine kinases associated sensory extracellular) domain family (Anantharaman & Aravind, Trends Biochem. Sci 26:579-582 (2001)). 3D homology searches with the present results, however, revealed significant structural similarities between AHK4 and bacterial family 1 HK sensor domains (Zhang & Hendrickson, J. Mol. Biol. 400:335-353 (2010)) (FIG. 3). The cytoplasmic segments of these bacterial HKs are functional dimers (Gao & Stock, Annu. Rev. Microbiol. 63:133-154 (2009); Marina et al., EMBO J. 24:4247-4259 (2005)), while the isolated sensor domains often are monomers in solution. Dimers of AHK4 are detected in both the crystal lattice and in size exclusion chromatography (FIG. 1a, FIG. 4c). The structurally related HK family 1 sensor domains form very similar crystallographic dimers along the N-terminal stalk helix (FIG. 3b-e). The dimerization interface in AHK4 (˜1,900 Å²) is highly conserved among the known plant cytokinin receptors (FIG. 4a, and FIG. 5a, b). Mutations in this interface inhibit dimer formation in solution and, importantly, render the receptor constitutively active (FIG. 4b, c and FIG. 5a, b). AHK4 may be a constitutive homodimer, as the Asp262Arg mutation in the hormone-binding pocket that likely interferes with cytokinin perception and receptor activity, does not effect the propensity of AHK4 to dimerize (FIG. 1c and FIG. 5c). The present findings indicate that plant cytokinin receptors are not activated by ligand-induced dimerization, but instead share a common activation mechanism with bacterial family 1 sensor HKs.

Different activation mechanisms have been proposed for these family 1 sensors, including ligand-induced piston movements of the transmembrane helices. In the present structure, the cytokinin binding pocket is closed by a ‘lid’, a short loop segment that directly contacts the hormone (FIG. 1b and FIG. 2). Mutation of ‘lid’ residues 280-284 to alanine render the receptor inactive (Heyl et al., BMC Evol. Biol. 7:62 (2007)) (FIG. 1b, c). Upon ligand binding, the ‘lid’ likely reorients and causes small structural changes in the underlying PAS domain. Indeed we find ‘lid’ Arg282 in contact with G1u326, which is located in a ‘transmitter’ helix connecting the membrane-distal and membrane-proximal PAS domains. Mutation of both residues to alanine inactivates AHK4 (FIG. 6a, c).

In contrast, mutation of Leu333 (a gain-of-function mutation in L. japonicus HK1²⁵) or Va1329 in the ‘transmitter’ helix into Phe, renders the receptor constitutively active (FIG. 6a, c) by inducing structural changes in the membrane-proximal PAS domain that are similar to a ligand-induced movement of the ‘transmitter’ helix. Va1329 and Leu333 are in close proximity to the disulfide bridge that links the second PAS domain to the N-terminal stalk helix (FIG. 6b). This disulfide likely acts as a hinge that translates local structural changes into a movement of the very C-terminal β-strand in AHK4. This would create a piston-like and/or lateral movement of TM2 with respect to TM2′ in the dimer partner, thereby signaling the loading state of the sensor to the cytoplasmic kinase domain (FIG. 6a and FIG. 7). Consistent with this model, several missense mutations in TM2 itself render AHK4 constitutively active, as does disruption of the dimer interface (FIG. 7 and above). The present results show that receptor activation in AHK4 is similar to the mechanism suggested for bacterial family 1 sensors. This model for activation is supported by the present complex structures with an endogenous ligand, mutational analyses, and a set of consistent genetic findings (Mähönen, et al.; Tirichine et al., Science 315:104-107 (2007); Miwa et al.).

### D. Example 3

### Comparison of AHK4 Bound to N⁶-isopentenyl adenine (iP) and N⁶-benzyladenine (BA)

The ability of AHK4 to perceive chemically diverse natural and synthetic cytokinins (Romanov et al., J. Exp. Bot. 57:4051-4058 (2006)) was next investigated (FIG. 8), by displacing iP in the binding pocket with an excess of cytokinin ligand during purification. A complex structure with N⁶-benzyladenine (BA) reveals that the hormone binding pocket can accommodate both isoprenoid and aromatic tail groups with no major structural rearrangements (FIG. 9a, b and Table 2). Next, we determined a 1.5 Å complex structure with trans-zeatin (tZ) (Table 2), the most potent endogenous cytokinin in Arabidopsis. By comparison with the iP and BA bound structures, the hydroxylated isopentenyl side chain of tZ establishes an additional hydrogen bond with Thr294 (FIG. 9c). The present structure, with only Thr294 as a hydrogen bond acceptor in the binding pocket, shows why AHK4 specifically recognises trans but not cis zeatin-type cytokinins with high affinity. Consistent with this, a complex structure with dihydrozeatin (DZ) reveals the hydroxyl group in the same orientation as found in the tZ complex (FIG. 8, FIG. 9c and Table 2).

### E. Example 4

### Structure of AHK4 with Synthetic cytokinin thidiazuron

Finally, we solved a structure with the synthetic cytokinin thidiazuron (TD), an important defoliant and herbicide. This structure shows that both natural and synthetic cytokinins occupy the same binding site in AHK4 (FIG. 9e). The phenyl moiety of TD binds to the tail pocket and the thiadiazol group mimics the adenine ring. (FIG. 9f). Importantly, both the urea-moiety of TD and the thiadiazol group establish polar interactions (with Asp262 and Leu284, respectively) that are similar to those observed in the adenine-type complexes (FIG. 9f). The present TD-bound structure shows why the serendipitously discovered urea derivatives (Amasino, Plant Physiol. 138:1177-1184 (2005)) are potent cytokinins. Taken together, the different hormone complexes described here reveal a highly adaptable binding pocket in which most receptor-hormone interactions are mediated by small hydrophobic residues. Discriminating polar contacts are established by Asp262 and by Thr294, which controls for binding of the correct stereoisomer (FIG. 9c). The presently disclosed structures unravel basic design principles for active cytokinins, i.e. the presence of a planar ring structure that occupies the adenine binding pocket, followed by a linker competent to establish hydrogen bonds with Asp262 and a planar aliphatic or aromatic tail group (FIG. 9). The rational design of novel synthetic cytokinins is thus now possible.

The TD bound structure is consistent with that of the classic cytokinin Kinetin (Miller et al., Journal of the American Chemical Society 78:1375-1380 (1956)) (KIN, Table 2), which shows that the ligand binding pocket can accommodate larger and charged tail groups. Again, Thr294 is involved in a polar interaction with the furfuryl group of KIN, in this case mediated by a water molecule (FIG. 9d).

In planta, cytokinins can be modified by N-glucosylation and N-alanine conjugation on the adenine ring and by O-glucosylation and O-acetylation on the isoprenoid tail for transport and storage (Bajguz & Piotrowska, Phytochemistry 70:957-969 (2009)). These modifications can render the plant cytokinin inactive. This agrees with our structures that reveal the N³ and N⁹, and especially the N⁷ position of the adenine ring buried in the binding pocket (FIG. 10). In addition, the shape and predominantly apolar nature of the tail pocket restricts binding of larger tail groups as found in tZ O-acetyl and tZ O-glucoside (FIG. 10).

In summary, the present disclosure defines the molecular basis for the perception of natural and synthetic cytokinins and establishes a strong evolutionary link between bacterial and eukaryotic HKs. The structural and mechanistic similarities between the AHK4 sensor domain and bacterial family 1 sensors, and the presence of iP in bacterial cells (Caillet & Droogmans, J. Bacteriol. 170:4147-4152 (1988); Gray et al., Plant Physiol. 110:431-438 (1996)), indicate that cytokinin signaling pathways evolved in bacteria and were later adopted to control developmental processes in plants and other eukaryotes (Anjard & Loomis, Development 135:819-827 (2008)).

### Informal Sequence Listing

AHK4 (Arabidopsis)
(SEQ ID NO: 1)
MNWALNNHQEEEEEPRRIEISDSESLENLKSSDFYQLGGGGALNSSEKPRKIDFWRSGLMGFAKMQQQQQ
LQHSVAVKMNNNNNNDLMGNKKGSTFIQEHRALLPKALILWIIIVGFISSGIYQWMDDANKIRREEVLVS
MCDQRARMLQDQFSVSVNHVHALAILVSTFHYHKNPSAIDQETFAEYTARTAFERPLLSGVAYAEKVVNF
EREMFERQHNWVIKTMDRGEPSPVRDEYAPVIFSQDSVSYLESLDMMSGEEDRENILRARETGKAVLTSP
FRLLETHHLGVVLTFPVYKSSLPENPTVEERIAATAGYLGGAFDVESLVENLLGQLAGNQAIVVHVYDIT
NASDPLVMYGNQDEEADRSLSHESKLDFGDPFRKHKMICRYHQKAPIPLNVLTTVPLFFAIGFLVGYILY
GAAMHIVKVEDDFHEMQELKVRAEAADVAKSQFLATVSHEIRTPMNGILGMLAMLLDTELSSTQRDYAQT
AQVCGKALIALINEVLDRAKIEAGKLELESVPFDIRSILDDVLSLFSEESRNKSIELAVFVSDKVPEIVK
GDSGRFRQIIINLVGNSVKFTEKGHIFVKVHLAEQSKDESEPKNALNGGVSEEMIVVSKQSSYNTLSGYE
AADGRNSWDSFKHLVSEEQSLSEFDISSNVRLMVSIEDTGIGIPLVAQGRVFMPFMQADSSTSRNYGGTG
IGLSISKCLVELMRGQINFISRPHIGSTFWFTAVLEKCDKCSAINHMKKPNVEHLPSTFKGMKAIVVDAK
PVRAAVTRYHMKRLGINVDVVTSLKTAVVAAAAFERNGSPLPTKPQLDMILVEKDSWISTEDNDSEIRLL
NSRTNGNVHHKSPKLALFATNITNSEFDRAKSAGFADTVIMKPLRASMIGACLQQVLELRKTRQQHPEGS
SPATLKSLLTGKKILVVDDNIVNRRVAAGALKKFGAEVVCAESGQVALGLLQIPHTFDACFMDIQMPQMD
GFEATRQIRMMEKETKEKTNLEWHLPILAMTADVIHATYEECLKSGMDGYVSKPFEEENLYKSVAKSFKP
NPISPSS

TABLE 1
Crystallographic data collection, phasing, and refinement of the AHK4
sensor domain in complex with iP that co-purified from E. coli
AHK4¹²⁶⁻³⁹⁵-iP

| Data collection | | | |
|---|---|---|---|
| Space group; unit cell (Å, °) | P32 2 1; 60.1, 60.1, 298.1, 90, 90, 120 | | |
| | KI soak | LiCl soak | Native |
| Wavelength (Å) | 1.5418 | 1.5418 | 0.9998 |
| Resolution (Å) | 2.74 | 2.35 | 1.65 |
| Highest shell (Å) | 2.91-2.74 | 2.41-2.35 | 1.69-1.65 |
| # Unique reflections* | 17,321 (2,562) | 26,786 (1,715) | 76,048 (5,521) |
| Multiplicity | 19.9 (19.9) | 7.3 (3.5) | 10.4 (8.8) |
| I/σ(I) | 21.1 (6.1) | 26.6 (8.4) | 30.6 (2.9) |
| R_{sym} (%)^{†} | 12.9 (43.9) | 5.3 (11.5) | 4.2 (70.3) |
| Completeness (%) | 99.9 (95.5) | 98.2 (85.0) | 100.0 (100.0) |
| Phasing | | | |
| Figure of merit [symbol] | | 0.70 (0.38) | |
| Refinement | | | |
| Resolution range (Å) | | | 19.8-1.65 |
| Highest shell (Å) | | | 1.69-1.65 |
| R [symbol] (%) [symbol] | | | 17.6 (25.8) |
| R [symbol] (%) [symbol] | | | 20.1 (26.8) |
| # atoms | | | |
| protein/solvent | | | 4,205/543 |
| iP/malonate | | | 30/7 |
| Average B factor (Å²) | | | |
| protein/solvent | | | 25.7/37.6 |
| iP/malonate | | | 17.7/28.2 |
| Geometry | | | |
| r.m.s.d. bonds (Å) | | | 0.012 |
| r.m.s.d. angles (°) | | | 1.36 |

[symbol]

Numbers in parentheses provide respective statistics for the highest resolution shell [symbol]
As defined in XDS³² [symbol]
As defined in RESOLVE³⁵, after and before density modification and phase extension [symbol]
As defined in REFMAC5³⁸ [symbol]
indicates data missing or illegible when filed

TABLE 2
Crystallographic data collection and refinement of various natural and synthetic
cytokinin complexes

| | | | tZr | | | |
|---|---|---|---|---|---|---|
| | tZ | DZ | (hydrolysed) | BA | KIN | TD |
| Data collection | | | | | | |
| Space group; | P 32 2 1; 59.8, 59.8, | P 32 2 1; 60, 60, | P 32 2 1; 59.9, 59.9, | P 32 2 1; 60.1, 60.1, | P 32 2 1; 59.9, 59.9, | P 32 2 1; 60.1,60.1, |
| unit cell (Å) | 297.3, 90, 90, 120 | 297.9, 90, 90, 120 | 297.4, 90, 90, 120 | 297.5, 90. 90, 120 | 297.9, 90, 90, 120 | 297.6, 90, 90, 120 |
| Wavelength (Å) | 0.9998 | 1.0331 | 1.5418 | 0.9998 | 0.9998 | 0.9998 |
| Resolution (Å) | 1.53 | 1.75 | 2.30 | 1.77 | 1.60 | 1.70 |
| Highest shell (Å) | 1.57-1.53 | 1.79-1.75 | 2.40-2.30 | 1.82-1.77 | 1.64-1.60 | 1.74-1.70 |
| # Unique reflections [symbol] | 94.704 (6.916) | 64.550 (4.596) | 28.135 (3.227) | 62.050 (4.122) | 84.408 (6.057) | 67.742 (4.853) |
| Multiplicity | 9.9 (8.3) | 8.6 (7.6) | 3.1 (2.7) | 10.8 (6.9) | 10.2 (6.8) | 11.0 (9.3) |
| I/σ(I)ⁱ | 19.8 (2.73) | 20.2 (2.0) | 14.9 (5.3) | 17.0 (2.8) | 25.6 (2.2) | 28.27 (3.0) |
| R [symbol] (%) [symbol] | 5.7 (64.7) | 6.7 (92.2) | 5.4 (19.8) | 8.9 (49.5) | 4.8 (81.9) | 4.6 (83.2) |
| Completeness (%) | 100.0 (100.0) | 99.7 (97.8) | 97.5 (95.9) | 99.4 (91.6) | 100 (99.9) | 96.4 (94.8) |
| Refinement | | | | | | |
| Resolution range (Å) | 19.82-1.53 | 19.69-1.75 | 29.36-2.30 | 46.11-1.77 | 19.59-1.60 | 19.68-1.70 |
| Highest shell (Å) | 1.57-1.53 | 1.79-1.75 | 2.38-2.30 | 1.82-1.77 | 1.64-1.60 | 1.74-1.70 |
| R [symbol] (%) [symbol] | 18.0 (25.8) | 18.0 (26.4) | 18.0 (19.1) | 18.0 (25.0) | 17.8 (27.5) | 18.3 (26.4) |
| R [symbol] (%) [symbol] | 20.0 (30.3) | 20.2 (31.0) | 23.7 (28.8) | 21.0 (30.1) | 20.0 (28.7) | 20.1 (28.7) |
| # of atoms | | | | | | |
| protein/solvent | 4.210/575 | 4.219/543 | 4.210/432 | 4227/514 | 4.206/549 | 4.223/512 |
| cytokinin/malonate | 32/7 | 32/7 | 32/7 | 34/7 | 32/7 | 30/7 |
| Average B factor (Å [symbol] ) | | | | | | |
| protein/solvent | 27.0/37.9 | 27.2/37.5 | 29.6/38.5 | 27.2/37.7 | 26.3/38.3 | 30.9/40.3 |
| cytokinin/malonate | 18.1/26.9 | 20.7/32.3 | 15.8/35.1 | 27.9/29.6 | 19.7/30.9 | 22.8/26.9 |
| r.m.s.d. bonds (Å) | 0.011 | 0.013 | 0.002 | 0.014 | 0.012 | 0.014 |
| r.m.s.d. angles ( [symbol] ) | 1.38 | 1.36 | 0.55 | 1.39 | 1.42 | 1.48 |

[symbol]

Numbers in parentheses provide respective statistics for the highest resolution shell
ⁱAs defined in XDS [symbol] , [symbol] As defined in REFMAC5 [symbol] [symbol]
indicates data missing or illegible when filed

1. An isolated protein comprising a 3-dimensional crystal structure of Arabidopsis Histidine Kinase 4 (AHK4) sensor domain being structurally defined by the atomic coordinate data shown in Tables 1 and 2.

2. An isolated protein comprising a 3-dimensional structure of the Arabidopsis Histidine Kinase 4 (AHK4) sensor domain, with a space group P32 2 1 and unit cell dimensions a=60.0±0.2 angstrom, b=60.0±0.2 angstrom, c=297.8±0.5, with alpha=90, beta=90, and gamma=120.

3. An isolated protein comprising a 3-dimensional structure of the Arabidopsis Histidine Kinase 4 (AHK4) sensor domain being structurally defined by the diagrams shown in FIGS. 1, 2, and 9.

4. The protein of claim 1, wherein the protein comprises a sequence having at least 90% identity to residues 126-391 of SEQ ID NO:1.

5. The protein of claim 1, wherein the protein binds to cytokinins selected from the group consisting of: N⁶-isopentyl adenine, N⁶-benzyladenine, trans-zeatin, kinetin, and thidiazuron.

6. A method of identifying a candidate modulator of Arabidopsis Histidine Kinase 4 (AHK4), comprising
(a) comparing the structure of a test compound with the structure of AHK4, said AHK4 comprising a 3 dimensional structure selected from the group consisting of:
(i) a sensor domain structurally defined by the atomic coordinate data shown in Tables 1 and 2;
(ii) a space group P32 2 1 and unit cell dimensions a=60.0±0.2 angstrom, b=60.0±0.2 angstrom, c=297.8±0.5, with alpha=90, beta=90, and gamma=120; and
(iii) a sensor domain structurally defined by the diagrams shown in FIGS. 1, 2, and 9;
(b) determining whether the test compound is likely to interact with AHK4; and
(c) identifying a candidate AHK4 modulator when the test compound in step (b) is determined to be likely to interact with AHK4.

7. The method of claim 6, further comprising validating the candidate AHK4 modulator by contacting the candidate AHK4 modulator with AHK4 and detecting interaction of the candidate AHK4 modulator with AHK4.

8. The method of claim 6, further comprising detecting an effect of the candidate AHK4 modulator when contacted with a AHK4 expressing plant, wherein the effect is selected from the group consisting of:
speeding leaf senescence,
delaying leaf senescence,
speeding seed germination,
delaying seed germination,
increasing shoot and root length, and
reducing shoot and root length,
as compared to a standard control.

9. The method of claim 6, wherein the candidate AHK4 modulator interacts with the ligand-binding region of AHK4.

10. The method of claim 6, wherein the candidate AHK4 modulator interacts with the dimerization region of AHK4.

11. A method of identifying a candidate modulator of Arabidopsis Histidine Kinase 4 (AHK4), comprising
(a) contacting a test compound with AHK4, said AHK4 comprising a 3 dimensional structure selected from the group consisting of:
(i) a sensor domain structurally defined by the atomic coordinate data shown in Tables 1 and 2;
(ii) a space group P32 2 1 and unit cell dimensions a=60.0±0.2 angstrom, b=60.0±0.2 angstrom, c=297.8±0.5, with alpha=90, beta=90, and gamma=120; and
(iii) a sensor domain structurally defined by the diagrams shown in FIGS. 1, 2 and 9; and
(b) detecting interaction of the test compound with AHK4, thereby identifying a candidate modulator of AHK4.

12. The method of claim 11, further comprising detecting an effect of the candidate AHK4 modulator when contacted with a AHK4 expressing plant, wherein the effect is selected from the group consisting of:
speeding leaf senescence,
delaying leaf senescence,
speeding seed germination,
delaying seed germination,
increasing shoot and root length, and
reducing shoot and root length,
as compared to a standard control.

13. The method of claim 11, wherein the candidate AHK4 modulator interacts with the ligand binding region of AHK4.

14. The method of claim 11, wherein the candidate AHK4 modulator interacts with the dimerization region of AHK4.

15. The protein of claim 2, wherein the protein comprises a sequence having at least 90% identity to residues 126-391 of SEQ ID NO:1.

16. The protein of claim 2, wherein the protein binds to cytokinins selected from the group consisting of: N⁶-isopentyl adenine, N⁶-benzyladenine, trans-zeatin, kinetin, and thidiazuron.

17. The protein of claim 3, wherein the protein comprises a sequence having at least 90% identity to residues 126-391 of SEQ ID NO:1.

18. The protein claim 3, wherein the protein binds to cytokinins selected from the group consisting of: N⁶-isopentyl adenine, N⁶-benzyladenine, trans-zeatin, kinetin, and thidiazuron.
